(19) United States
(12) Patent Application Publication
(10) Pub. No.: US 2023/0148952 A1
(43) Pub. Date: May 18, 2023

(54) SYSTEMS AND METHODS FOR ANALYZING SLEEP-RELATED PARAMETERS

(71) Applicants: ResMed Asia Pte. Ltd., Singapore (SG); ResMed Pty Ltd, Bella Vista NSW (AU); ResMed Sensor Technologies Limited, Dublin (IE); ResMed (Malaysia) Sdn. Bhd., Kuala Lumpur (MY)

(72) Inventors: Christopher Andrew Wakeley Gill, Singapore (SG); Alvin Lim Zhi Guang, Shah Alam, Selangor (MY); Sarah Terese McGann, Sydney (AU); Timothy Mark Stephenson, Queensland (AU); Matthew James Shaw, Sydney (AU); Michael Wren, Dublin (IE); Redmond Shouldice, Dublin (IE); Kieran Conway, Dublin (IE); Michael John Costello, Dublin (IE); Stephen Dodd, Dublin (IE); Cesar Lopes, Dublin (IE); Gaetano Caldarola, Bella Vista NSW (AU)

(21) Appl. No.: 17/919,891

(22) PCT Filed: Apr. 20, 2021
(86) PCT No.: PCT/IB2021/053225

§ 371 Date: Oct. 19, 2022

Related U.S. Application Data
(60) Provisional application No. 63/012,869, filed on Apr. 20, 2020.
(60) Provisional application No. 63/151,507, filed on Feb. 19, 2021.

(51) Int. Cl. A61B 5/00; A61B 7/00; G16H 20/40

(52) U.S. Cl. CPC A61B 5/4818; A61B 7/003; A61B 5/4815; A61B 5/7282; A61B 5/4848; G16H 20/40; A61M 16/0003

(57) ABSTRACT

A method includes receiving first data associated with a first sleep session of a user. The method also includes determining a first set of sleep-related parameters associated with the first sleep session of the user based at least in part on the first data. The method also includes receiving second data associated with a second sleep session of the user. The method also includes determining a second set of sleep-related parameters associated with the second sleep session of the user based at least in part on the second data. The method also includes receiving third data associated with a variable condition. The method also includes causing one or more indications associated with the variable condition and the first sleep session, the second sleep session, or both to be communicated to the user.

### CROSS-REFERENCE TO RELATED APPLICATIONS

This application claims the benefit of, and priority to, U.S. Provisional Patent Application No. 63/012,869, filed on Apr. 20, 2020, and U.S. Provisional Patent Application No. 63/151,507, filed on Feb. 19, 2021, both of which are hereby incorporated by reference herein in their entirety.

### TECHNICAL FIELD

The present disclosure relates generally to systems and methods for determining one or more sleep-related parameters for a plurality of sleep sessions, and more particularly, to systems and methods for comparing one or more sleep-related parameters associated with a first sleep session and one or more sleep-related parameters associated with a second sleep session.

### BACKGROUND

Many individuals suffer from sleep-related and/or respiratory disorders such as, for example, Periodic Limb Movement Disorder (PLMD), Restless Leg Syndrome (RLS), Sleep-Disordered Breathing (SDB), Obstructive Sleep Apnea (OSA), Respiratory Effort Related Arousal (RERA), Central Sleep Apnea (CSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. These disorders are often treated using a respiratory therapy system. However, some users find such systems to be uncomfortable, difficult to use, expensive, aesthetically unappealing and/or fail to perceive the benefits associated with using the system. As a result, some users will discontinue use of the respiratory therapy system absent encouragement or affirmation that the respiratory therapy system is improving their sleep quality and reducing the symptoms of these disorders. The present disclosure is directed to solving these and other problems.

### SUMMARY

According to some implementations of the present disclosure, a method includes receiving first data associated with a first sleep session of a user. The method also includes determining a first set of sleep-related parameters associated with the first sleep session of the user based at least in part on the first data. The method also includes receiving second data associated with a second sleep session of the user. The method also includes determining a second set of sleep-related parameters associated with the second sleep session of the user based at least in part on the second data. The method also includes receiving third data associated with a variable condition. The method also includes causing one or more indications associated with the variable condition and the first sleep session, the second sleep session, or both to be communicated to the user.

According to some implementations of the present disclosure, a method includes receiving physiological data associated with a user. The method also includes determining (i) a first emotion score associated with the user, (ii) a sleepiness level associated with the user, or both (i) and (ii) based at least in part on the physiological data. The method also includes causing a prompt to interact with a therapy system to be communicated to the user based at least in part on the emotion score, the sleepiness level, or both.

According to some implementations of the present disclosure, a method includes receiving, from one or more sensors, first data associated with a first sleep session of a user, the first data including (i) first respiration data associated with the user, (ii) first audio data reproducible as one or more sounds recorded during the first sleep session, or (iii) both (i) and (ii), wherein the user did not use a respiratory therapy system during the first sleep session. The method also includes determining a first set of sleep-related parameters associated with the first sleep session of the user based at least in part on the first data. The method also includes receiving, from the one or more sensors, second data associated with a second sleep session of the user, the second data including (i) second respiration data associated with the user, (ii) second audio data reproducible as one or more sounds recorded during the second sleep session, or (iii) both (i) and (ii), wherein the user used the respiratory therapy system during at least a portion of the second sleep session. The method also includes determining a second set of sleep-related parameters associated with the second sleep session of the user based at least in part on the second data. The method also includes causing one or more indications associated with the first sleep session, the second sleep session, or both, to be communicated to the user, via a user device, subsequent to the second sleep session.

According to some implementations of the present disclosure, a system includes a respiratory therapy system, a memory storing machine-readable instructions, and a control system. The respiratory therapy system includes a respiratory device configured to supply pressurized air and a user interface coupled to the respiratory device via a conduit, the user interface being configured to engage a user and aid in directing the supplied pressurized air to an airway of the user. The control system includes one or more processors configured to execute the machine-readable instructions to receive first data generated by one or more sensors and associated with a first sleep session of a user, wherein the user did use a respiratory therapy system during the first sleep session. The control system is further configured to determine a first set of sleep-related parameters associated with the first sleep session for the user based at least in part on the first data. The control system is further configured to receive, from the one or more sensors, second data associated with a second sleep session of the user, wherein the user interface of the respiratory therapy system engaged the user during at least a portion of the second sleep session. The control system is further configured to determine a second set of sleep-related parameters associated with the second sleep session for the user based at least in part on the second data. The control system is further configured to cause one or more indications associated with the first sleep session, the second sleep session, or both to be communicated to the user via the display of the user device subsequent to the second sleep session.

According to some implementations of the present disclosure, a method includes receiving, from one or more sensors, first data associated with a first sleep session of a user, the first data including (i) first respiration data associated with the user, (ii) first audio data reproducible as one or more sounds recorded during the first sleep session, or (iii) both (i) and (ii), wherein the user did not use a respiratory therapy system during the first sleep session. The method also includes determining a first set of sleep-related parameters associated with the first sleep session of the user based at least in part on the first data, the first set of sleep-related parameters including a first apnea-hypopnea index (AHI) for the first sleep session and determining a first sleep condition for the first sleep session based at least in part on the first AHI. The method also includes receiving, from the one or more sensors, second data associated with a second sleep session of the user, the second data including (i) second respiration data associated with the user, (ii) second audio data reproducible as one or more sounds recorded during the second sleep session, or (iii) both (i) and (ii), wherein the user used the respiratory therapy system during at least a portion of the second sleep session. The method also includes determining a second set of sleep-related parameters associated with the second sleep session of the user based at least in part on the second data, the second set of sleep-related parameters including a second AHI for the second sleep session, determining a first sleep condition for the second sleep session based at least in part on the second AHI, and causing one or more indications of (i) the first sleep condition, (ii) the second sleep condition, or (iii) both (i) and (ii) to be communicated to the user via a user device subsequent to the first sleep session.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3 illustrates an exemplary timeline for a sleep session, according to some implementations of the present disclosure;

FIG. 4 illustrates an exemplary hypnogram associated with the sleep session of FIG. 3, according to some implementations of the present disclosure;

FIG. 5A illustrates an exemplary setup view for manually initiating a sleep session, according to some implementations of the present disclosure;

FIG. 5B illustrates an exemplary sleep view for manually terminating a sleep session, according to some implementations of the present disclosure;

FIG. 5C illustrates an exemplary alarm view for terminating a sleep session, according to some implementations of the present disclosure;

FIG. 6 is a process flow diagram for a method of comparing a first sleep session and a second sleep session, according to some implementations of the present disclosure;

FIG. 7A illustrates a first prompt for providing subjective feedback associated with a sleep session, according to some implementations of the present disclosure;

FIG. 7B illustrates a second prompt for providing subjective feedback associated with a first sleep session, according to some implementations of the present disclosure;

FIG. 7C illustrates a third prompt for providing subjective feedback associated with the first sleep session, according to some implementations of the present disclosure;

FIG. 7D illustrates a fourth prompt for providing subjective feedback associated with the first sleep session, according to some implementations of the present disclosure;

FIG. 8A illustrates a first plurality of indications associated with the first sleep session, according to some implementations of the present disclosure;

FIG. 8B illustrates a second plurality of indications associated with the first sleep session, according to some implementations of the present disclosure;

FIG. 8C illustrates a third plurality of indications associated with the first sleep session, according to some implementations of the present disclosure;

FIG. 9A illustrates a prompt for providing subjective feedback associated with use of a respiratory therapy system during a second sleep session, according to some implementations of the present disclosure;

FIG. 9B illustrates a first plurality of indications associated with the second sleep session, according to some implementations of the present disclosure;

FIG. 9C illustrates a second plurality of indications associated with the second sleep session, according to some implementations of the present disclosure;

FIG. 10 illustrates a comparison between one or more sleep-related parameters of the second sleep session and one or more sleep-related parameters of the second sleep session, according to some implementations of the present disclosure;

FIG. 11 is a process flow diagram for a method according to some implementations of the present disclosure;

FIG. 12 is a process flow diagram for a method according to some implementations of the present disclosure;

FIG. 13A is a first sleep label view according to some implementations of the present disclosure;

FIG. 13B is a second sleep label view according to some implementations of the present disclosure;

FIG. 13C is a third sleep label view according to some implementations of the present disclosure;

FIG. 14 is an activity reporting view according to some implementations of the present disclosure;

FIG. 15 is a sleep dashboard according to some implementations of the present disclosure;

FIG. 16A is a first trend view according to some implementations of the present disclosure; and

FIG. 16B is a second trend view according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB), Obstructive Sleep Apnea (OSA), Central Sleep Apneas (CSA), and other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). Other types of apneas include hypopnea, hyperpnoea, and hypercapnia. Hypopnea is generally characterized by slow or shallow breathing caused by a narrowed airway, as opposed to a blocked airway. Hyperpnoea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events must fulfil both of the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in International Patent Publication No. WO 2008/138040 and U.S. Patent Publication No. 2011/0203588, assigned to ResMed Ltd., the disclosures of which are hereby incorporated by reference herein in their entirety.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping. While these other sleep-related disorders may have similar symptoms as insomnia, distinguishing these other sleep-related disorders from insomnia is useful for tailoring an effective treatment plan distinguishing characteristics that may call for different treatments. For example, fatigue is generally a feature of insomnia, whereas excessive daytime sleepiness is a characteristic feature of other disorders (e.g., OSA) and reflects a physiological propensity to fall asleep unintentionally.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

Many individuals also suffer from insomnia, a condition which is generally characterized by a dissatisfaction with sleep quality or duration (e.g., difficulty initiating sleep, frequent or prolonged awakenings after initially falling asleep, and an early awakening with an inability to return to sleep). It is estimated that over 2.6 billion people worldwide experience some form of insomnia, and over 750 million people worldwide suffer from a diagnosed insomnia disorder. In the United States, insomnia causes an estimated gross economic burden of $107.5 billion per year, and accounts for 13.6% of all days out of role and 4.6% of injuries requiring medical attention. Recent research also shows that insomnia is the second most prevalent mental disorder, and that insomnia is a primary risk factor for depression.

Comorbid insomnia refers to a type of insomnia where the insomnia symptoms are caused at least in part by a symptom or complication of another physical or mental condition (e.g., anxiety, depression, medical conditions, and/or medication usage). Mixed insomnia refers to a combination of attributes of other types of insomnia (e.g., a combination of sleep-onset, sleep-maintenance, and late insomnia symptoms). Paradoxical insomnia refers to a disconnect or disparity between the user's perceived sleep quality and the user's actual sleep quality.

Nocturnal insomnia symptoms generally include, for example, reduced sleep quality, reduced sleep duration, sleep-onset insomnia, sleep-maintenance insomnia, late insomnia, mixed insomnia, and/or paradoxical insomnia. Sleep-onset insomnia is characterized by difficulty initiating sleep at bedtime. Sleep-maintenance insomnia is characterized by frequent and/or prolonged awakenings during the night after initially falling asleep. Late insomnia is characterized by an early morning awakening (e.g., prior to a target or desired wakeup time) with the inability to go back to sleep.

Diurnal (e.g., daytime) insomnia symptoms include, for example, fatigue, reduced energy, impaired cognition (e.g., attention, concentration, and/or memory), difficulty functioning in academic or occupational settings, and/or mood disturbances. These symptoms can lead to psychological complications such as, for example, lower performance, decreased reaction time, increased risk of depression, and/or increased risk of anxiety disorders. Insomnia symptoms can also lead to physiological complications such as, for example, poor immune system function, high blood pressure, increased risk of heart disease, increased risk of diabetes, weight gain, and/or obesity. Insomnia can also be categorized based on its duration. For example, insomnia symptoms are typically considered acute or transient if they occur for less than 3 months. Conversely, insomnia symptoms are typically considered chronic or persistent if they occur for 3 months or more, for example. Persistent/chronic insomnia symptoms often require a different treatment path than acute/transient insomnia symptoms.

Mechanisms of insomnia include predisposing factors, precipitating factors, and perpetuating factors. Predisposing factors include hyperarousal, which is characterized by increased physiological arousal during sleep and wakefulness. Measures of hyperarousal include, for example, increased levels of cortisol, increased activity of the autonomic nervous system (e.g., as indicated by increase resting heart rate and/or altered heart rate), increased brain activity (e.g., increased EEG frequencies during sleep and/or increased number of arousals during REM sleep), increased metabolic rate, increased body temperature and/or increased activity in the pituitary-adrenal axis. Precipitating factors include stressful life events (e.g., related to employment or education, relationships, etc.) Perpetuating factors include excessive worrying about sleep loss and the resulting consequences, which may maintain insomnia symptoms even after the precipitating factor has been removed.

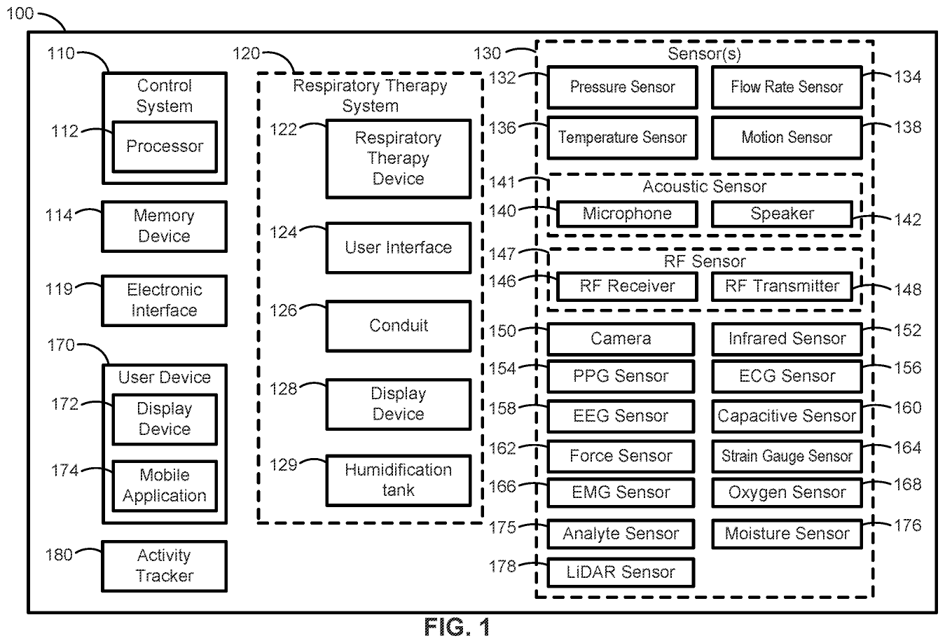

Referring to FIG. 1, a system 100, according to some implementations of the present disclosure, is illustrated. The system 100 includes a control system 110, a memory device 114, an electronic interface 119, one or more sensors 130, and one or more user devices 170. In some implementation, the system 100 further optionally includes a respiratory therapy system 120 and/or an activity tracker 180.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory therapy device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological data and/or audio data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a Wi-Fi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 optionally includes a respiratory therapy system 120 (also referred to as a respiratory therapy system). The respiratory therapy system 120 can include a respiratory pressure therapy device 122 (referred to herein as respiratory therapy device 122), a user interface 124, a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory therapy device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory therapy device 122 is generally used to generate pressurized air that is delivered to a user (e.g., using one or more motors that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory therapy device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory therapy device 122 to the user's airway to aid in preventing the airway from narrowing and/or collapsing during sleep. This may also increase the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

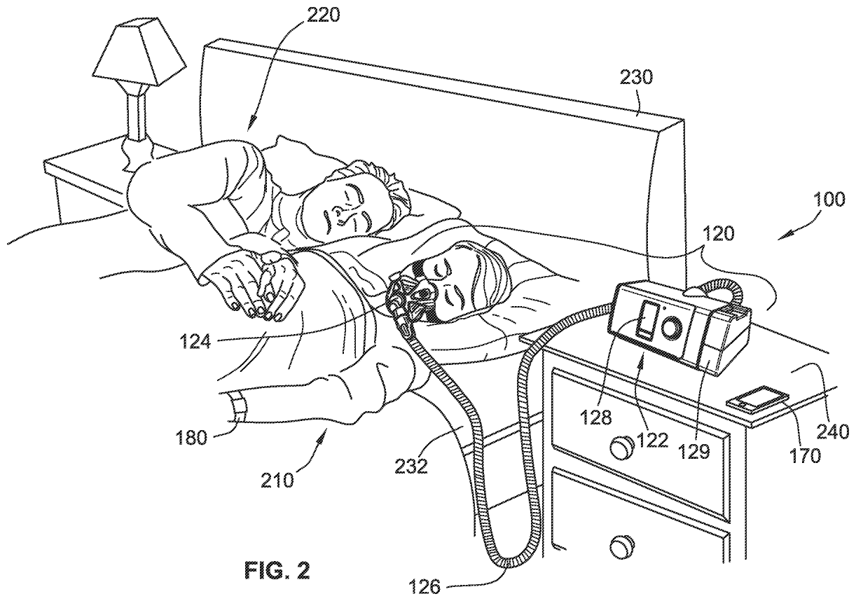

As shown in FIG. 2, in some implementations, the user interface 124 is a facial mask that covers the nose and mouth of the user. Alternatively, the user interface 124 can be a nasal mask that provides air to the nose of the user or a nasal pillow mask that delivers air directly to the nostrils of the user. The user interface 124 can include a plurality of straps (e.g., including hook and loop fasteners) for positioning and/or stabilizing the interface on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 includes a mouthpiece (e.g., a night guard mouthpiece molded to conform to the user's teeth, a mandibular repositioning device (MRD), etc.).

The conduit 126 (also referred to as an air circuit or tube) allows the flow of air between two components of a respiratory therapy system 120, such as the respiratory therapy device 122 and the user interface 124. In some implementations, there can be separate limbs of the conduit for inhalation and exhalation. In other implementations, a single limb conduit is used for both inhalation and exhalation.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be use, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score or a therapy score (also referred to as a myAir™ score, such as described in WO 2016/061629 and U.S. Patent Publication No. 2017/0311879, which are hereby incorporated by reference herein in their entirety), the current date/time, personal information for the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122 and includes a reservoir of water that can be used to humidify the pressurized air delivered from the respiratory therapy device 122. The respiratory therapy device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user.

The respiratory therapy system 120 can be used, for example, as a positive airway pressure (PAP) system, a continuous positive airway pressure (CPAP) system, an automatic positive airway pressure system (APAP), a bi-level or variable positive airway pressure system (BPAP or VPAP), a ventilator, or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory therapy system 120 and a bed partner 220 are located in a bed 230 and are laying on a mattress 232. The user interface 124 (e.g., a full facial mask) can be worn by the user 210 during a sleep session. The user interface 124 is fluidly coupled and/or connected to the respiratory therapy device 122 via the conduit 126. In turn, the respiratory therapy device 122 delivers pressurized air to the user 210 via the conduit 126 and the user interface 124 to increase the air pressure in the throat of the user 210 to aid in preventing the airway from closing and/or narrowing during sleep. The respiratory therapy device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

As described herein, the system 100 generally can be used to generate physiological data associated with a user (e.g., a user of the respiratory therapy system 120 shown in FIG. 2) during a sleep session. The physiological data can be analyzed to generate one or more sleep-related parameters, which can include any parameter, measurement, etc. related to the user during the sleep session. The one or more sleep-related parameters that can be determined for the user 210 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 122, a heart rate, a heart rate variability, movement of the user 210, temperature, EEG activity, EMG activity, arousal, snoring, choking, coughing, whistling, wheezing, or any combination thereof.

The one or more sensors 130 can be used to generate, for example, physiological data, audio data, or both. Physiological data generated by one or more of the sensors 130 can be used by the control system 110 to determine a sleep-wake signal associated with a user during a sleep session and one or more sleep-related parameters. The sleep-wake signal can be indicative of one or more sleep states and/or sleep stages, including wakefulness, relaxed wakefulness, micro-awakenings, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 130, are described in, for example, International Patent Publication No. WO 2014/047310, U.S. Patent Publication No. 2015/0230750, U.S. Patent Publication No. 2014/0088373, WO 2017/132726, WO 2019/122413, and WO 2019/122414, and U.S. Patent Publication No. 2020/383580, each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 130 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 122, or any combination thereof during the sleep session. The event(s) can include snoring, apneas, central apneas, obstructive apneas, mixed apneas, hypopneas, a mask leak (e.g., from the user interface 124), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 130 can also be used to determine a respiration signal associated with a user during a sleep session. The respiration signal is generally indicative of respiration or breathing of the user during the sleep session. The respiration signal can be indicative of, for example, a respiration rate, a respiration rate variability, an inspiration amplitude, an expiration amplitude, an inspiration-expiration ratio, a number of events per hour, a pattern of events, pressure settings of the respiratory therapy device 122, or any combination thereof. The event(s) can include snoring, apneas, central apneas, obstructive apneas, mixed apneas, hypopneas, a mask leak (e.g., from the user interface 124), a restless leg, a sleeping disorder, choking, an increased heart rate, labored breathing, an asthma attack, an epileptic episode, a seizure, or any combination thereof.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory therapy device 122. The pressure sensor 132 can be, for example, a capacitive sensor, an electromagnetic sensor, a piezoelectric sensor, a strain-gauge sensor, an optical sensor, a potentiometric sensor, or any combination thereof.

The flow rate sensor 134 outputs flow rate data that can be stored in the memory device 114 and/or analyzed by the processor 112 of the control system 110. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the respiratory therapy device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user 210 during the sleep session, and/or detect movement of any of the components of the respiratory therapy system 120, such as the respiratory therapy device 122, the user interface 124, or the conduit 126. The motion sensor 138 can include one or more inertial sensors, such as accelerometers, gyroscopes, and magnetometers. In some implementations, the motion sensor 138 alternatively or additionally generates one or more signals representing bodily movement of the user, from which may be obtained a signal representing a sleep state of the user; for example, via a respiratory movement of the user. In some implementations, the motion data from the motion sensor 138 can be used in conjunction with additional data from another sensor 130 to determine the sleep state of the user.

The microphone 140 outputs sound and/or audio data that can be stored in the memory device 114 and/or analyzed by the processor 112 of the control system 110. The audio data generated by the microphone 140 is reproducible as one or more sound(s) during a sleep session (e.g., sounds from the user 210). The audio data form the microphone 140 can also be used to identify (e.g., using the control system 110) an event experienced by the user during the sleep session, as described in further detail herein. The microphone 140 can be coupled to or integrated in the respiratory therapy device 122, the use interface 124, the conduit 126, or the user device 170. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones.

The speaker 142 outputs sound waves that are audible to a user of the system 100 (e.g., the user 210 of FIG. 2). The speaker 142 can be used, for example, as an alarm clock or to play an alert or message to the user 210 (e.g., in response to an event). In some implementations, the speaker 142 can be used to communicate the audio data generated by the microphone 140 to the user. The speaker 142 can be coupled to or integrated in the respiratory therapy device 122, the user interface 124, the conduit 126, or the user device 170.

The microphone 140 and the speaker 142 can be used as separate devices. In some implementations, the microphone 140 and the speaker 142 can be combined into an acoustic sensor 141, as described in, for example, International Patent Publication Nos. WO 2018/050913 and WO 2020/104465, each of which is hereby incorporated by reference herein in its entirety. In such implementations, the speaker 142 generates or emits sound waves at a predetermined interval and/or frequency and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, pressure settings of the respiratory therapy device 122, or any combination thereof. In this context, a sonar sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO2018/050913 and WO 2020/104465 mentioned above.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory therapy device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147. In some such implementations, the RF sensor 147 includes a control circuit. The specific format of the RF communication can be Wi-Fi, Bluetooth, or the like.

In some implementations, the RF sensor 147 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 147. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or any combination thereof) that can be stored in the memory device 114. The image data from the camera 150 can be used by the control system 110 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 150 can be used to, for example, identify a location of the user, to determine chest movement of the user 210 (FIG. 2), to determine air flow of the mouth and/or nose of the user 210, to determine a time when the user 210 enters the bed 230 (FIG. 2), and to determine a time when the user 210 exits the bed 230. In some implementations, the camera 150 includes a wide angle lens or a fish eye lens.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state or sleep stage of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, a pulse oximeter (e.g., SpO₂ sensor), or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the breath of the user 210. In some implementations, the analyte sensor 174 is positioned near a mouth of the user 210 to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory therapy device 122, etc.). Thus, in some implementations, the moisture sensor 176 can be coupled to or integrated in the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user 210, for example, the air inside the bedroom.

The Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 130 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the user device 170, the activity tracker 180, or any combination thereof. For example, the microphone 140 and speaker 142 is integrated in and/or coupled to the user device 170 and the pressure sensor 130 and/or flow rate sensor 132 are integrated in and/or coupled to the respiratory therapy device 122. In some implementations, at least one of the one or more sensors 130 is not coupled to the respiratory therapy device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The user device 170 (FIG. 1) includes a display device 172. The user device 170 can be, for example, a mobile device such as a smartphone, a tablet, a gaming console, a smart watch, a laptop, or the like. Alternatively, the user device 170 can be an external sensing system, a television (e.g., a smart television) or another smart home device (e.g., a smart speaker(s) such as Google Home®, Amazon Echo®, Amazon Alexa etc.). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

In some implementations, the user device 170 is a smartphone and includes the display device 172, one or more processors (e.g., that are the same as, or similar to the processor 112), one or more memory devices (e.g., that are the same as, or similar to, the memory device 114), and one or more of the sensors 130 (e.g., the microphone 140, the speaker 142, the RF receiver 146, the RF transmitter 148, and the camera 150). In other implementations, the user device 170 is a smart speaker or hub (e.g., that is the same as, or similar to, Google Home®, Google Nest®, Google Nest Hub®, Amazon Echo®, Amazon Alexa, etc.) that includes the display device 172, one or more processors (e.g., that are the same as, or similar to the processor 112), one or more memory devices (e.g., that are the same as, or similar to, the memory device 114), and one or more of the sensors 130 (e.g., the microphone 140, the speaker 142, the RF receiver 146, the RF transmitter 148). In such implementations, the sensor(s) included in the user device 170 can be used to generate or obtain the data associated with a sleep session (e.g., physiological data, audio data, etc.) described herein. In some implementations, the user device is a wearable device (e.g., a smart watch).

In some implementations, the user device 170 includes a mobile application 174 for executing and/or providing a user interface for any of the methods described herein. The mobile application 174 can be downloaded to the user device 170 from an application store or pre-installed on the user device 170 (e.g., as part of the native operating system).

In some implementations, the system 100 also includes an activity tracker 180. The activity tracker 180 is generally used to aid in generating physiological data associated with the user. The activity tracker 180 can include one or more of the sensors 130 described herein, such as, for example, the motion sensor 138 (e.g., one or more accelerometers and/or gyroscopes), the PPG sensor 154, and/or the ECG sensor 156. The physiological data from the activity tracker 180 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 180 is coupled (e.g., electronically or physically) to the user device 170.

In some implementations, the activity tracker 180 is a wearable device that can be worn by the user, such as a smartwatch, a wristband, a ring, or a patch. For example, referring to FIG. 2, the activity tracker 180 is worn on a wrist of the user 210. The activity tracker 180 can also be coupled to or integrated a garment or clothing that is worn by the user. Alternatively, the activity tracker 180 can also be coupled to or integrated in (e.g., within the same housing) the user device 170. More generally, the activity tracker 180 can be communicatively coupled with, or physically integrated in (e.g., within a housing), the control system 110, the memory 114, the respiratory therapy system 120, and/or the user device 170. In other words, the activity tracker 180 can sync with the control system 110, the memory 114, the respiratory therapy system 120, and/or the user device 170.

Referring back to FIG. 1, while the control system 110 and the memory device 114 are described and shown in FIG. 1 as being a separate and distinct component of the system 100, in some implementations, the control system 110 and/or the memory device 114 are integrated in the user device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof. In some implementations, some aspects of the processing described herein are carried out in the cloud which advantageously allows updating of physiological data and/or sleep-related parameters.

While system 100 is shown as including all of the components described above, more or fewer components can be included in a system for generating physiological data and determining a recommended notification or action for the user according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and the user device 170. Thus, various systems can be formed using any portion or portions of the components shown and described herein and/or in combination with one or more other components.

As used herein, a sleep session can be defined in a number of ways based on, for example, an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 172 of the user device 170 (FIG. 1) to manually initiate or terminate the sleep session.

Generally, the sleep session includes any point in time after the user 210 has laid or sat down in the bed 230 (or another area or object on which they intend to sleep), and has turned on the respiratory therapy device 122 and donned the user interface 124. The sleep session can thus include time periods (i) when the user 210 is using the CPAP system but before the user 210 attempts to fall asleep (for example when the user 210 lays in the bed 230 reading a book); (ii) when the user 210 begins trying to fall asleep but is still awake; (iii) when the user 210 is in a light sleep (also referred to as stage 1 and stage 2 of non-rapid eye movement (NREM) sleep); (iv) when the user 210 is in a deep sleep (also referred to as slow-wave sleep (SWS), or stage 3 of NREM sleep); (v) when the user 210 is in rapid eye movement (REM) sleep; (vi) when the user 210 is periodically awake between light sleep, deep sleep, or REM sleep; or (vii) when the user 210 wakes up and does not fall back asleep.

In some examples, the sleep session can be generally defined as ending once the user 210 removes the user interface 124, turns off the respiratory therapy device 122, and gets out of bed 230. In some implementations, the sleep session can include additional periods of time, or can be limited to only some of the above-disclosed time periods. For example, the sleep session can be defined to encompass a period of time beginning when the respiratory therapy device 122 begins supplying the pressurized air to the airway or the user 210, ending when the respiratory therapy device 122 stops supplying the pressurized air to the airway of the user 210, and including some or all of the time points in between, when the user 210 is asleep or awake.

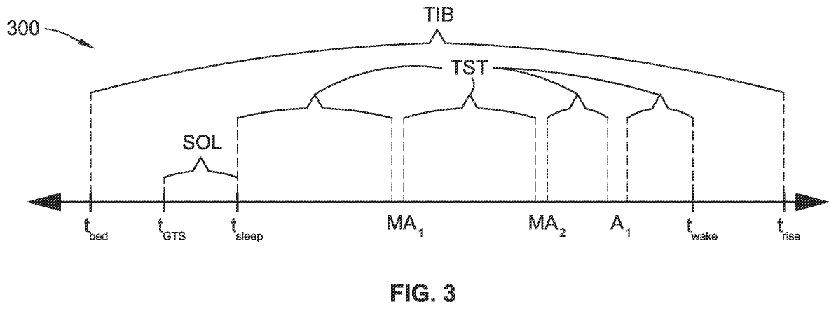

Referring to FIG. 3, an exemplary timeline 300 for a sleep session is illustrated. The timeline 300 includes an enter bed time (t_{bed}), a go-to-sleep time (t_{GTS}), an initial sleep time (tₛₗₑₑₚ), a first micro-awakening MA₁, a second micro-awakening MA₂, an awakening A₁, a wake-up time (t_{wake}), and a rising time (tᵣᵢₛₑ).

The enter bed time t_{bed} is associated with the time that the user initially enters the bed (e.g., bed 230 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (t_{GTS}) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 170, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A₁) after initially falling asleep (e.g., getting up to go to the bathroom, attending to children or pets, sleep walking, etc.). However, the user goes back to sleep after the awakening A₁. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 300 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

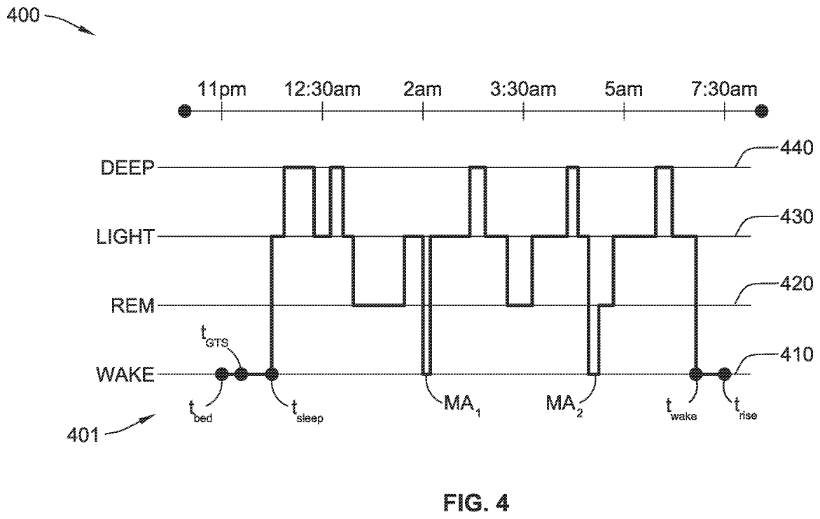

Referring to FIG. 4, an exemplary hypnogram 400 corresponding to the timeline 300 (FIG. 3), according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based on physiological data associated with the user (e.g., generated by one or more of the sensors 130 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 114.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 130 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 138, the microphone 140, the camera 150, or any combination thereof. The go-to-sleep time can be determined based on, for example, data from the motion sensor 138 (e.g., data indicative of no movement by the user), data from the camera 150 (e.g., data indicative of no movement by the user and/or that the user has turned off the lights) data from the microphone 140 (e.g., data indicative of the using turning off a TV), data from the user device 170 (e.g., data indicative of the user no longer using the user device 170), data from the pressure sensor 132 and/or the flow rate sensor 134 (e.g., data indicative of the user turning on the respiratory therapy device 122, data indicative of the user donning the user interface 124, etc.), or any combination thereof.

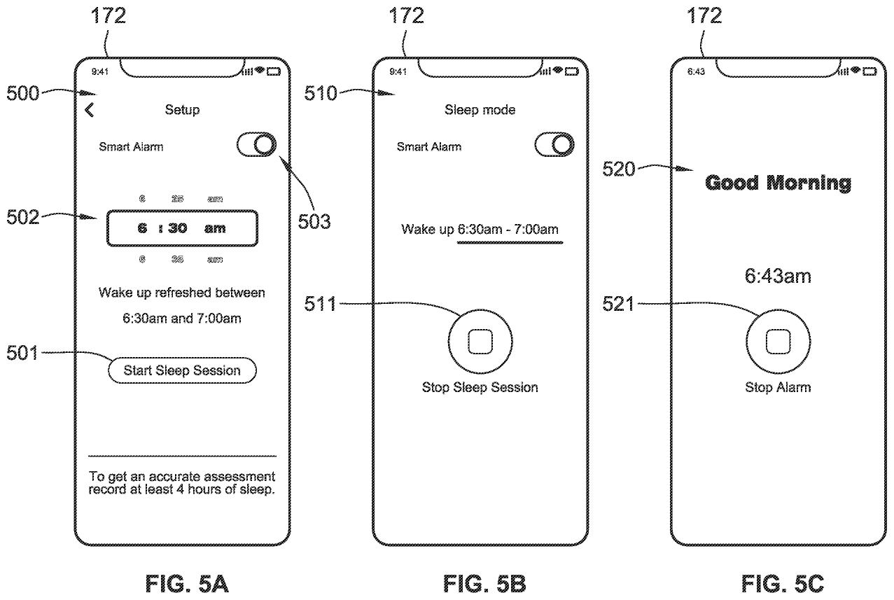

Referring generally to FIGS. 5A-5C, in some implementations, the user can manually define the beginning of a sleep session and/or terminate a sleep session. Referring to FIG. 5A, a setup view 500 is displayed via the display device 172 of the user device 170 (FIG. 1). The setup view 500 includes a first user-selectable element 501, a second user-selectable element 502, and a third user-selectable element 503. The first user-selectable element 501 allows the user to define the start of a sleep session (e.g., by clicking or tapping the first user-selectable element 501). The second user-selectable element 502 allows the user to specify a wake-up time (e.g., 6:30 AM). The second user-selectable element 502 can include a scroll wheel menu or a dropdown menu, for example, to allow the user to specify the wake-up time. The third user-selectable element 503 allows the user to select (e.g., with a toggle button) a smart alarm feature that will generate an alarm based on the selected wake-up time.

Generally, the smart alarm can generate an alarm within a predetermined time range (e.g., between 6:30 AM and 7:00 AM) based on the selected wake-up time, and generate the alarm at an optimal time within that range based on physiological data and/or sleep-related parameters. For example, the smart alarm can generate an alarm within a predetermined time window (e.g., within 15 minutes, 20 minutes, 30 minutes, 45 minutes, 1 hours, etc.) relative to a desired wakeup time in which the user is closest to light sleep (as determined based on one or more sleep-related parameters, as described herein). To illustrate, if the predetermined time window is 30 minutes and the desired wake-up is 7:00 AM, the smart alarm can generate an alarm at time between 6:30 AM and 7:00 AM when the user is closest to light sleep.

Referring to FIG. 5B, a sleep view 510 is displayed on the display device 172 of the user device 170 (FIG. 1). The sleep view 510 is displayed subsequent to (e.g., in response to) the user selecting the first user-selectable element 501 of the setup view 500 (FIG. 5A). The sleep view 510 (FIG. 5B) includes a first user-selectable element 511 that permits the user to manually terminate the sleep session (e.g., by clicking or tapping the first user-selectable element 511).

Referring to FIG. 5C, an alarm view 520 is displayed on the display device 172 of the user device 170 (FIG. 1). The alarm view 520 is displayed subsequent to (e.g., in response to) the alarm generated via the second user-selectable element 502 of the setup view 500 (FIG. 5A). The alarm view 520 includes a first user-selectable element 521 that permits the user to turn off the alarm (e.g., by clicking or tapping the first user-selectable element 521). As shown, in this example, the alarm is generated at 6:43 AM, which is within the predetermined range (between 6:30 AM and 7:00 AM) set by the smart alarm feature described above.

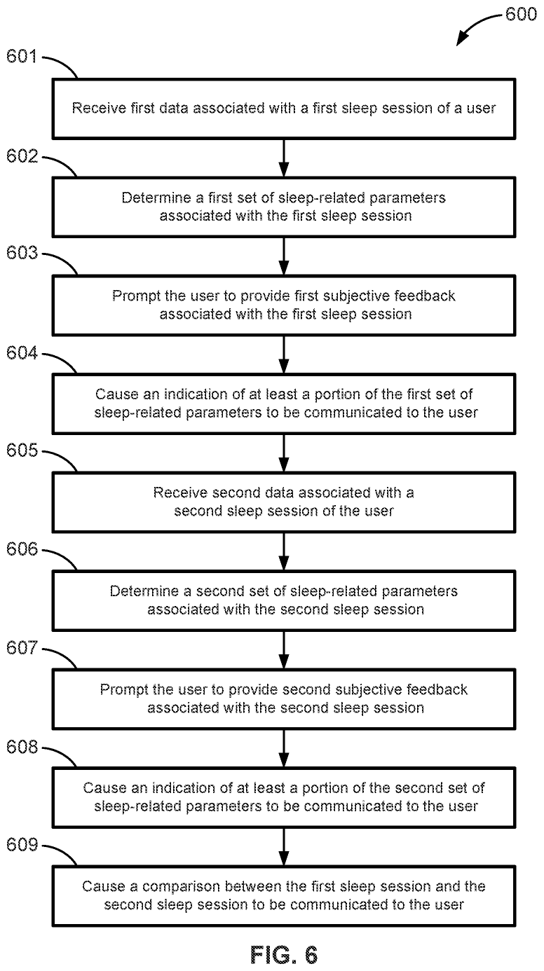

Referring to FIG. 6, a method 600 for comparing a first sleep condition for a first sleep session of a user and a second sleep condition for a second sleep session of the user is illustrated. As described herein, the second sleep session differs from the first sleep session in that the user uses a respiratory therapy system (e.g., that is the same as, or similar to, the respiratory therapy system 120 described herein) during at least a portion of the second sleep session, but does not use the respiratory therapy system during the first sleep session. One or more steps of the method 600 can be implemented using any element or aspect of the system 100 (FIGS. 1-2) described herein.

Step 601 of the method 600 includes generating and/or receiving first data associated with a first sleep session of a user. The user does not use a respiratory therapy system (e.g., that is the same as, or similar to, the respiratory therapy system 120 described herein) during the first sleep session when the first data is generated or obtained. The first data (step 601) can be generated by, for example, one or more of the sensors 130 (FIG. 1) described herein, including the acoustic sensor 141.

The first data can include, for example, first respiration data associated with the user, first audio data associated with the user, or both. The first respiration data is indicative of a first respiration signal of the user during at least a portion of the first sleep session (e.g., at least 10% of the first sleep session, at least 50% of the first sleep session, 75% of the first sleep session, at least 90% of the first sleep session, etc.). The respiration signal is indicative of a respiration rate, a respiration rate variability, a tidal volume, an inspiration amplitude, an expiration amplitude, an inspiration-expiration ratio, etc., or any combination thereof of the user during at least a portion of the first sleep session. The first audio data is reproducible as one or more sounds recorded during the first sleep session (e.g., snoring, choking, labored breathing, etc.).

In some implementations, both the first respiratory data and the first audio data are generated by the acoustic sensor 141 (FIG. 1), where the acoustic sensor is coupled to or embedded in user device 170. In other implementations, the first respiratory data is generated by the acoustic sensor 141, whereas the first audio data is generated by a microphone (e.g., that is the same as, or similar to, the microphone 140) that is separate and distinct from the acoustic sensor. The first data can be received by, for example, the electronic interface 119 and/or the user device 170 (FIG. 1) described herein.

In some implementations, the first data received during step 601 can include sleep-label data. Generally, the sleep label data is indicative of the user's use of a product (e.g., respiratory therapy system or device) during the first sleep session. In such implementations, the first data received during step 601 can be associated with the selected sleep label.

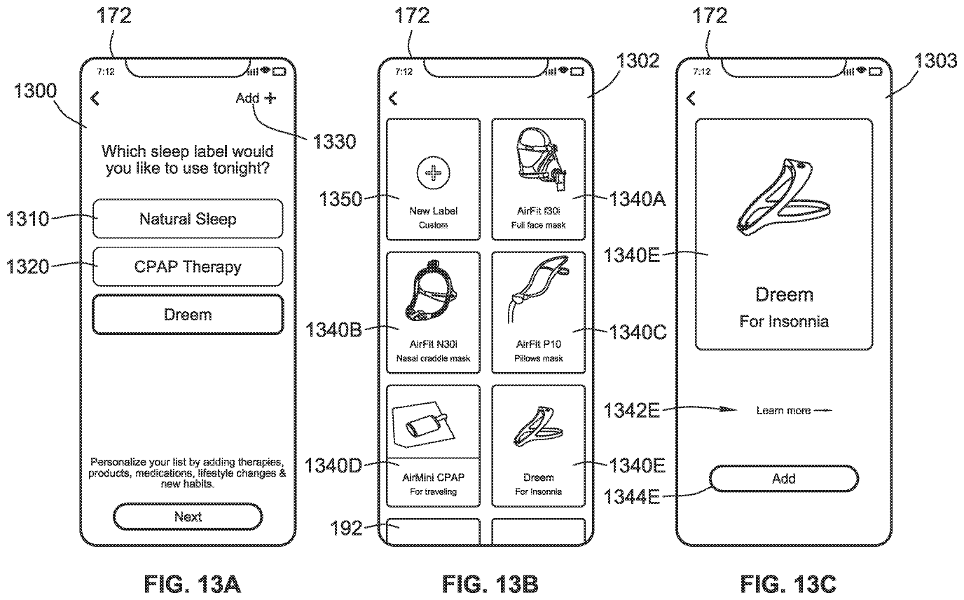

For example, referring to FIG. 13A, a first sleep label view 1300 can be displayed on the display device 172 prior to the first sleep session. The first sleep label view 1300 can include a first user-selectable element 1310 and a second user-selectable element 1320. In the exemplary implementation shown in FIG. 13A, the user can select the first user-selectable element 1310 to apply a first sleep label (e.g., that is indicative of no use of a respirator therapy system or device during the first sleep session). Alternatively, the user can select the second user-selectable element 1320 to apply a second sleep label (e.g., that is indicative of use a respiratory therapy system or device during the first sleep session). The selection of the first sleep label or second sleep label via the first sleep label view 1300 is associated with the at least a portion of the other first data that is received during step 601.

In some implementations, the first sleep label view 1300 further includes a third user-selectable element 1314 that can be selected by the user to create cause another user-selectable element associated with a third sleep label (e.g., a third sleep label that is different than the first sleep label associated with the first user-selectable element 1312 and different than the second sleep label associated with the second user-selectable element 1314) to be displayed within the first sleep label view 1300. For example, referring to FIG. 13B, a selection of the third user-selectable element 1316 in the first sleep label view 1300 causes a second sleep label view 1302 to be displayed on the display device 172. Generally, the second sleep label view 1302 permits a user to select from a plurality of predetermined sleep labels that will be added as an option in the first sleep label view 1300 (FIG. 13A) or create a custom sleep label.

In the example shown in FIG. 13B, the second sleep label view 1302 includes a plurality of user-selectable elements 1340A-1340E, each of which is associated with one of a plurality of predetermined sleep labels. For example, the first user-selectable element 1340A can be associated with a first user interface type for a respiratory therapy system (e.g., a full face mask), the second user-selectable element 1340B can be associated with a second user interface type for a respiratory therapy system (e.g., a nasal cradle mask), the third user-selectable element 1340C can be associated with a third user interface type for a respiratory therapy system (e.g., a pillow mask), and the fourth user-selectable element 1340D can be associated with a type of respiratory therapy system (e.g., a travel version) that is different than the respiratory therapy system associated with the first user-selectable element 1310 in the first sleep label view 1300 (FIG. 13A). The fifth user-selectable element 1340E can be associated with a therapy system (e.g., for insomnia) that is different than the respiratory therapy system associated with the first user-selectable element 1310 in the first sleep label view 1300 (FIG. 13A). Each of the user-selectable elements 1340A-1340E can include alphanumeric text and/or images associated with the corresponding device or system. The second sleep label view 1302 can also include a sixth user-selectable element 1350 for creating a custom sleep label (e.g., the user can input the custom sleep label using alphanumeric text). In other implementations, additional sleep labels can include use of a complementary device (e.g., pillow, bedding, sleep blanket, etc.) or a medication label (e.g., which medication the user used prior to a sleep session). In some implementations, the complementary device is a smart pillow, such as the ones described in International Application No. PCT/US2020/048633 (International Publication No. WO 2021/041987) and/or International Application No. PCT/IB2020/061244, both of which are hereby incorporated by reference herein in their entirety.

In some implementations, a third sleep label view can be displayed responsive to a selection of one of the plurality of user-selectable elements 1340A-1340E in the second sleep label view 1302 (FIG. 13B). For example, referring to FIG. 13C, a third sleep label view 1303 is displayed on the display device 172 responsive to a selection of the fifth user-selectable element 1340E. The third sleep label view 1303 includes the same or similar alphanumeric text and/or images as the fifth user-selectable element 1340E (e.g., an enlarged version of the fifth user-selectable element 1340E) and another user-selectable element 1342E that can be selected to provide more information associated with the insomnia therapy device (e.g., alphanumeric text, a link to a web page, an instruction manual, etc.). The user can cause another user-selectable element associated with the insomnia therapy device to be added to the first sleep label view 1300 (FIG. 13A) by selecting the user-selectable element 1344E.

In some implementations, a sleep label can be determined automatically and associated with the first data. For example, rather than the user manually indicating usage of a respiratory therapy system, step 601 can include automatically detecting that the user is using the respiratory therapy system (e.g., using one or more of the sensors 130 described herein). Further, in some examples, a therapy device type (e.g., type of user interface) can be automatically detected and applied as a sleep label. For instance, data from the camera 150 can be analyzed (e.g., using an object recognition algorithm) to identify the device of therapy device (e.g., type of user interface).

Step 602 of the method 600 includes determining a first set of sleep-related parameters associated with the first sleep session based at least in part on the first data generated and/or received during step 601. For example, the control system 110 can analyze the first data (e.g., that is stored in the memory device 114) to determine the first set of sleep-related parameters for the first sleep session. Information describing the determined first set of sleep-related parameters can be stored in the memory device 114 (FIG. 1), for example.

The first set of sleep-related parameters can include, for example, an apnea-hypopnea index (AHI), an identification of one or more events experienced by the user, a number of events per hour, a pattern of events, a total sleep time, a total time in bed, a wake-up time, a rising time, a hypnogram, a total light sleep time, a total deep sleep time, a total REM sleep time, a number of awakenings, a sleep-onset latency, or any combination thereof. In some implementations, the first set of sleep-related parameters can include a sleep score, such as the ones described in International Publication No. WO 2015/006364 and U.S. Patent Publication No. 2016/0151603, which are hereby incorporated by reference herein in their entirety. The first set of sleep-related parameters can include any number of sleep-related parameters (e.g., 1 sleep-related parameter, 2 sleep-related parameters, 5 sleep-related parameters, 50 sleep-related parameters, etc.).

In implementations where a sleep label (FIGS. 13A-13C) is selected prior to the first sleep session and is associated with the first data, the first set of sleep-related parameters can be associated with the sleep label.

Step 603 of the method 600 includes prompting the user to provide first subjective feedback associated with the first sleep session subsequent to the first sleep session. The first subjective feedback can be received by the user device 170 (e.g., via the display device 172) and stored in the memory device 114 (FIG. 1). For example, the user can be prompted to provide the first subsequent feedback immediately after clicking or tapping the first user selectable element 511 of the sleep view 510 (FIG. 5B) or the first user selectable element 521 of the alarm view 520 (FIG. 5C). More generally, the user can be prompted at any time subsequent to the first sleep session, but prior to a next, subsequent sleep session (e.g., 30 seconds after the first sleep session, 1 minute after the first sleep session, 5 minutes after the first sleep session, 15 minutes after the first sleep session, 30 minutes after the first sleep session, 1 hour after the first sleep session, 8 hours after the first sleep session, 12 hours after the first sleep session, etc.). The first subjective feedback can include, for example, a subjective sleepiness level subsequent to the first sleep session, a subjective sleepiness level prior to the first sleep session, a subjective sleep satisfaction rating for the first sleep session, or any combination thereof.

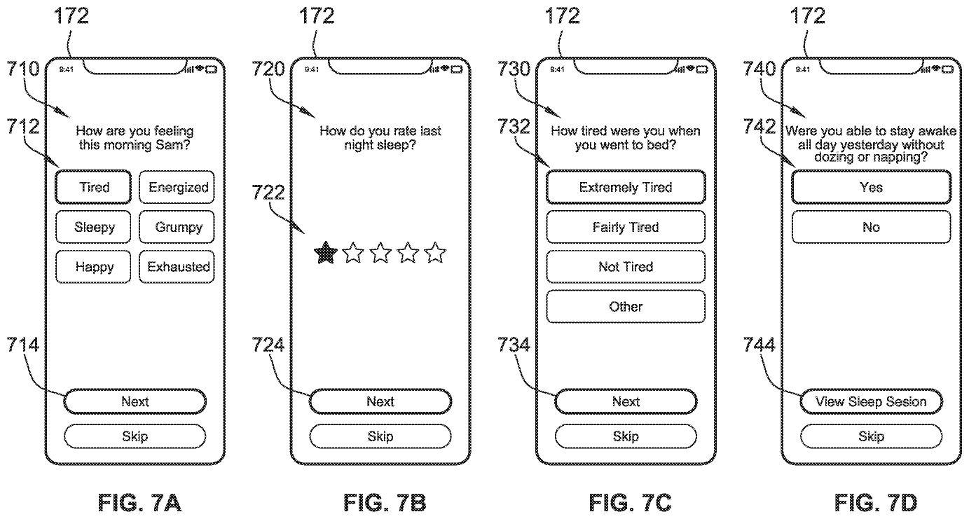

Information associated with or indicative of the first subjective feedback from the user can be received, for example, through the user device 170 (e.g., via alphanumeric text, speech-to-text, etc.). Referring generally to FIGS. 7A-7D, in some implementations, a series of successive prompts to the user for providing the first subjective feedback can be displayed on the display device 172 of the user device 170 (FIG. 1).

Referring to FIG. 7A, a first prompt 710 containing alphanumeric text prompts the user to indicate how they feel subsequent to the first sleep session using a plurality of user-selectable elements 712 (e.g., by clicking or tapping one or more of the elements 712). For example, the user can provide an indication of fatigue or sleepiness following the first sleep session. In the exemplary implementation shown in FIG. 7A, the plurality of user-selectable elements 712 include individual elements for indicating that the user is tired, energized, sleepy, grumpy, happy, exhausted, or any combination thereof. The user can finalize their selection of the elements 712 by selecting (e.g., clicking or tapping) navigation element 714 and proceed to the next prompt(s) (e.g., FIG. 7B).

Referring to FIG. 7B, a second prompt 720 containing alphanumeric text prompts the user to indicate a rating for the first sleep session. The rating can be generally indicative of the user's perceived quality of sleep for the first sleep session (e.g., poor, good, average, excellent, etc.). In the exemplary implementation shown in FIG. 7B, the second prompt 720 includes a user-selectable star input 722 for rating the first sleep session with 1 star, 2 stars, 3 stars, 4 stars, or 5 stars (with 5 stars being the best, 1 star being the worst). The user can finalize their star selection by selecting (e.g., clicking or tapping) navigation element 724 and proceed to the next prompt.

Referring to FIG. 7C, a third prompt 730 containing alphanumeric text prompts the user to indicate a sleepiness or fatigue level prior to initiating the first sleep session. The third prompt 730 includes a plurality of user-selectable elements 732 to permit the user to indicate a sleepiness or fatigue level prior to the first sleep session (e.g., extremely tired, fairly tired, not tired, other). In some implementations, the third prompt 730 can be provided to the user prior to the first sleep session (e.g., subsequent to selecting the first user selectable element 501 in the setup view 500 (FIG. 5A)).

Referring to FIG. 7D, a fourth prompt 740 containing alphanumeric text prompts the user to indicate whether they were able to stay awake during the day following the sleep session (e.g., 8 hours after the first sleep session, 12 hours after the first sleep session, 16 hours after the first sleep session, the entire time between the end of the first sleep session and a next sleep session etc.) without dozing or napping. The fourth prompt 740 includes a first plurality of user-selectable elements 742 for indicating “yes” or “no.” The user can finalize their selection by selecting navigation element 744 and view information associated with the first sleep session. Generally, the fourth prompt 740 can be displayed subsequent to the first sleep session, but prior to a second, subsequent sleep session (e.g., the next immediate sleep session after the first sleep session).

In some implementations, the subjective feedback can include activity information. The activity information can be associated with, for example, activity by the user prior to the first sleep session, or after the first sleep session and prior to a second sleep session. The activity information can be received before or after the first sleep session (e.g., a daily log). The activity information can include information associated with, exercise, naps, caffeine intake, alcohol intake, or any combination thereof. In some examples, the nap(s) are taken without using a therapy system (e.g., the respiratory therapy system described herein). In other examples, the nap(s) are taken using the therapy system. Taking a nap using the therapy system can aid the user in acclimating to using the therapy system in the future (e.g., at night).

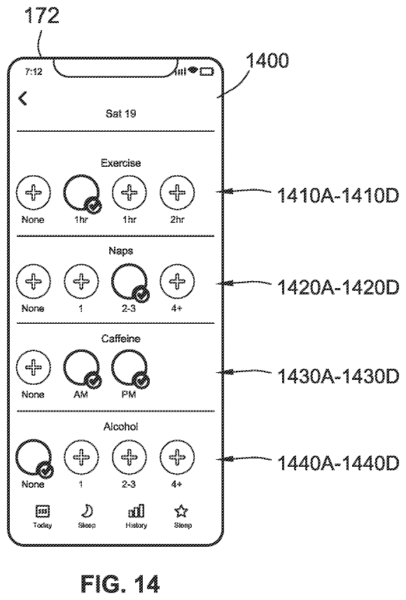

Referring to FIG. 14, in some implementations, an activity reporting view 1400 can be displayed on the display device 172. The activity reporting view 1400 includes a first plurality of user-selectable elements 1410A-1410D, a second plurality of user-selectable elements 1420A-1420D, a third plurality of user-selectable elements 1430A-1430C, and a fourth plurality of user-selectable elements 1440A-1440D.

The first plurality of user-selectable elements 1410A-1410D are associated with an amount of time the user exercised (e.g., prior to the first sleep session). By selecting a corresponding one of the first plurality of user-selectable elements 1410A-1410D, the user can indicate, for example, no exercise, more than 30 minutes or exercise, more than 1 hour of exercise, or more than 2 hours of exercise.

The second plurality of user-selectable elements 1420A-1420D are associated with a number of naps taken by the user (e.g., prior to the first sleep session). By selecting a corresponding one of the second plurality of user-selectable elements 1420A-1420D, the user can indicate, for example, no naps, 1 nap, between 2 and 3 naps, or more than 4 naps.

The third plurality of user-selectable elements 1430A-1430C are associated with a caffeine intake (e.g., prior to the first sleep session). By selecting a corresponding one of the third plurality of user-selectable elements 1410C-1410C, the user can indicate, for example, that the user consumed caffeine in the morning (AM), in the afternoon or evening (PM), or both.

The fourth plurality of user-selectable elements 1440A-1440D are associated with alcohol intake by the user (e.g., prior to the first sleep session). By selecting a corresponding one of the fourth plurality of user-selectable elements 1440A-1440D, the user can indicate, for example, no alcohol intake, 1 alcoholic beverage, 2 to 3 alcoholic beverages, or more than 4 alcoholic beverages (e.g., consumed prior to the first sleep session).

Step 604 of the method 600 (FIG. 6) includes causing an indication of at least a portion of the determined first set of sleep-related parameters (step 602) to be communicated to the user subsequent to the first sleep session. The indication(s) of the determined first set of sleep-related parameters can be communicated to the user via alphanumeric text, images, audio, or any combination thereof using, for example, the user device 170 (FIG. 1).

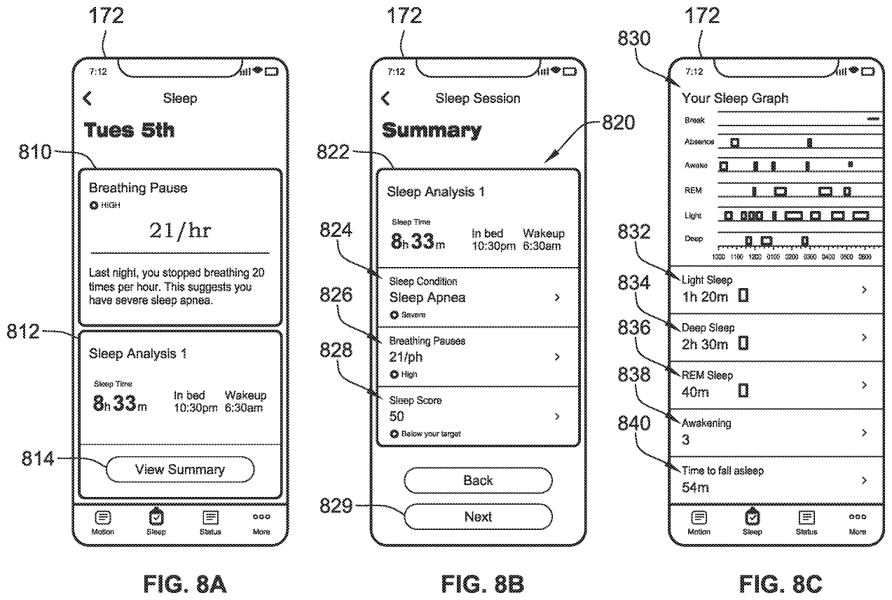

Referring to FIG. 8A, a plurality of indications 810-820 associated with the first sleep session are displayed on the display device 172 of the user device 170 (FIG. 1). In some implementations, the indications 810-820 can be displayed responsive to the user selecting (e.g., clicking or tapping) the navigation element 744 (FIG. 7D) described above. The plurality of indications includes an AHI indication 810 that includes information (e.g., alphanumeric text) indicative of a determined AHI for the first sleep session. As described herein, AHI is one of the sleep-related parameters that can be determined based on the first data associated with the first sleep session. The AHI indication 810 can include, for example, the determined AHI value (e.g., the numerical value) and/or a relative qualifier (e.g., low, moderate, high, etc.). In the example of FIG. 8A, the AHI value is 21 per hour and the relative qualifier is high. The AHI indication 810 can also include alphanumeric text explaining the AHI value and severity to the user (e.g., Last night, you stopped breathing 20 times per hour. This suggests you have severe sleep apnea).

The plurality of indications also includes a sleep analysis indication 812 including information indicative of a total sleep time, an enter bed time, a wake-up time, or any combination thereof for the first sleep session. In the example of FIG. 8A, the sleep analysis indication 812 includes alphanumeric text indicating that the total sleep time is 8 hours and 33 minutes, the enter bed time is 10:30 PM, and the wake-up time is 6:30 AM.

As shown in FIG. 8A, a sleep summary element 814 can also be displayed on the display device 172 for permitting the user to view a sleep summary associated with the first sleep session. Referring to FIG. 8B, a sleep summary 820 is displayed on the display device 172 subsequent (e.g., responsive to) to a selection of the sleep summary element 814 (FIG. 8A). The sleep summary 820 includes a sleep analysis indication 822, a sleep condition indication 824, an AHI indication 826, and a sleep score indication 828.

The sleep analysis indication 822 is the same as, or similar to, the sleep analysis indication 812 (FIG. 8A) described above and includes a total sleep time, an enter bed time, a wake-up time, or any combination thereof. The sleep condition indication 824 provides information indicative of whether the user experienced, for example, sleep apnea during the first sleep session, and the severity of the sleep apnea (e.g., mild, moderate, severe, etc.). In the example, of FIG. 8B, the sleep condition indication 824 indicates that the user experienced sleep apnea during the first sleep session, and that the sleep apnea was considered severe. The AHI indication 826 is the same as, or similar to, the AHI indication 810 described above (FIG. 8A). The sleep score indication 828 provides information indicative of a sleep score for the first sleep session and/or whether the determined sleep score meets or exceeds a target sleep score. The sleep score can be, for example, a myAir™ score, such as described in International Patent Publication No. WO 2016/061629 and U.S. Patent Publication No. 2017/0311879, which are incorporated by reference herein in their entirety. In the example of FIG. 8B, the sleep score indication 828 indicates that the sleep score for the first sleep session is 50, and that this score is below the target score for the user.

A navigation element 829 can also be displayed on the display device 172 along with the sleep summary 820 so that the user can view choose whether to view additional indications of the determined first set of sleep-related parameters for the first sleep session. Referring to FIG. 8C, subsequent to (e.g., responsive to) selecting the navigation element 829 (FIG. 8B), a plurality of indications 830-840 associated with the first sleep session are displayed on the display device 172. The plurality of indications 830-840 include a sleep graph 830, a light sleep indication 832, a deep sleep indication 834, a REM sleep indication 836, an awakening indication 838, and a sleep-onset latency indication 840.

The sleep graph 830 is a bar graph indicative of breaks during the sleep session (e.g., when the user is awake), absences during sleep session (e.g., when the user gets out of bed during the night), awakenings, REM sleep, light sleep, deep sleep, or any combination thereof. The light sleep indication 832 provides information indicative of a light sleep time for the first sleep session (in the example of FIG. 8C, 1 hour and 20 minutes). The deep sleep indication 834 provides information indicative of a deep sleep time for the first sleep session (in the example of FIG. 8C, 2 hours and 30 minutes). The REM sleep indication 836 provides information indicative of a REM sleep time for the first sleep session (in the example of FIG. 8C, 40 minutes). The awakening indication 838 provides information indicative of the number of awakenings during the first sleep session (in the example of FIG. 8C, 3 awakenings). The sleep-on-latency indication 840 provides information indicative of a sleep-onset latency for the first sleep session (in the example of FIG. 8C, 54 minutes).

In some implementations, step 604 includes causing an audio indication to be communicated to the user subsequent to the first sleep session. As described above, the first data (step 601) can include audio data reproducible as one or more sounds recorded during the first sleep session. During step 602, one or more events such as snoring, choking, labored breathing, or the like can be identified based at least in part on the first data. These events can be identified in the audio data based on, for example, a comparison between the audio data and previously recorded audio data (e.g., using a machine learning algorithm) or the audio data exceeded a predetermined decibel level. The audio data received during step 601 that includes these events can be stored in the memory device 114 (FIG. 1) for playback, while other audio data where no event is identified can be deleted to preserve storage capacity in the memory device 114.

While the noises associated with certain events like snoring, choking, or labored breathing can be quite loud (e.g., from the perspective of the bed partner 220 in FIG. 2), the user cannot hear these noises because they are asleep. If the user could hear these noises, they may be more likely or encouraged to seek treatment and use a respiratory therapy system in the future to reduce or eliminate these events. Thus, step 604 can include causing portions of the audio data corresponding to the events to be communicated to the user (e.g., using the speaker 142) so that the user can hear these events after the first sleep session.

In some implementations, step 604 includes causing an indication of a respiration signal for the first sleep session to be communicated to the user (e.g., via the display device 172 of the user device 170). The indication of the respiration signal can be, for example, a graph or plot. In such implementations, one or more indications of determined events associated with the first sleep session (e.g., apneas, snoring, choking, etc.) can be overlaid on the displayed respiration signal.

Referring back to FIG. 6, step 605 of the method 600 includes receiving second data associated with a second sleep session of the user that is subsequent to the first sleep session. The second sleep session differs from the first sleep session in that the user is using the respiratory therapy system 120 (FIG. 1) described herein during the second sleep session, but not the first sleep session. The second data can be received by, for example, the electronic interface 119 and/or the user device 170 (FIG. 1) described herein.

The second data (step 605) can be generated using the same sensor as the first data (step 601), or a different sensor or sensors. In some implementations, the first data (step 601) and the second data (step 605) are both generated by the acoustic sensor 141 (FIG. 1). In other implementations, the first data is generated by the acoustic sensor 141 that is coupled to or integrated in the user device 170, while the second data is generated by one or more of the sensors 130 described herein that is coupled to or integrated in the respiratory therapy device 122 (e.g., the pressure sensor 132, the flow rate sensor 134, or both). In still other implementations, the second data is generated by both the acoustic sensor 141 that is coupled to or integrated in the user device 170 and by one or more of the sensors 130 described herein that is coupled or integrated in the respiratory therapy device 122.

The second data is the same as, or similar to, the first data (step 601) and can include, for example, second respiration data associated with the user, second audio data associated with the user, or both. The second respiration data is indicative of is indicative of a second respiration signal of the user during at least a portion of the second sleep session (e.g., at least 10% of the second sleep session, at least 50% of the second sleep session, 75% of the second sleep session, at least 90% of the second sleep session, etc.). The respiration signal is indicative of a respiration rate, an inspiration amplitude, an expiration amplitude, an inspiration-expiration ratio, etc., or any combination thereof of the user during at least a portion of the second sleep session. The second audio data is reproducible as one or more sounds recorded during the second sleep session (e.g., snoring, choking, labored breathing, etc.). The second data (step 602) can be associated with one or more sleep labels (FIGS. 13A-13C) in the same or similar manner as the first data (step 601). For example, the method can include receiving a first sleep label associated with the first sleep session during step 601 and receiving a second sleep label that is different than the first sleep label during step 605 for the second sleep session.

In some implementations, the second sleep session (step 605) is the next immediate sleep session following the first sleep session (step 601) (e.g., the first sleep session is a Monday night and the second sleep session is a Tuesday night). In other implementations, there are one or more other sleep sessions between the first sleep session and the second sleep session (e.g., the first sleep session is on a Monday night and the second sleep session is the following Thursday night). The second sleep session can be manually initiated and/or terminated by the user in the same or similar manner as the first sleep session (FIGS. 5A-5C).

Step 606 of the method 600 includes determining a second set of sleep-related parameters associated with the second sleep session based at least in part on the second data generated and/or received during step 605. For example, the control system 110 can analyze the second data (e.g., that is stored in the memory device 114) to determine the second set of sleep-related parameters for the second sleep session. Information describing the determined second set of sleep-related parameters can be stored in the memory device 114 (FIG. 1), for example.

The second set of sleep-related parameters can include, for example, an apnea-hypopnea index (AHI), an identification of one or more events experienced by the user, a number of events per hour, a pattern of events, a sleep score, a total sleep time, a total time in bed, a wake-up time, a rising time, a hypnogram, a total light sleep time, a total deep sleep time, a total REM sleep time, a number of awakenings, a sleep-onset latency, a respiration rate, or any combination thereof. The second set of sleep-related parameters (step 606) can include the same parameters as the first set of sleep-related parameters (step 602), or different parameters. More generally, the second set of sleep-related parameters can include any number of sleep-related parameters (e.g., 1 sleep-related parameter, 2 sleep-related parameters, 5 sleep-related parameters, 50 sleep-related parameters, etc.).

Step 607 of the method 600 includes prompting the user to provide second subjective feedback associated with the second sleep session. Information associated with or indicative of the second subjective feedback from the user can be received, for example, through the user device 170 (e.g., via alphanumeric text, speech-to-text, etc.). The second subjective feedback can be received by the user device 170 (e.g., via the display device 172) and stored in the memory device 114 (FIG. 1). More generally, the user can be prompted at any time subsequent to the second sleep session, but prior to a next, subsequent sleep session (e.g., 30 seconds after the second sleep session, 1 minute after the second sleep session, 5 minutes after the second sleep session, 15 minutes after the second sleep session, 30 minutes after the second sleep session, 1 hour after the second sleep session, 8 hours after the second sleep session, 12 hours after the second sleep session, etc.). The second subjective feedback can include, for example, a subjective sleepiness level subsequent to the second sleep session, a subjective sleepiness level prior to the second sleep session, a subjective sleep satisfaction rating for the second sleep session, or any combination thereof.

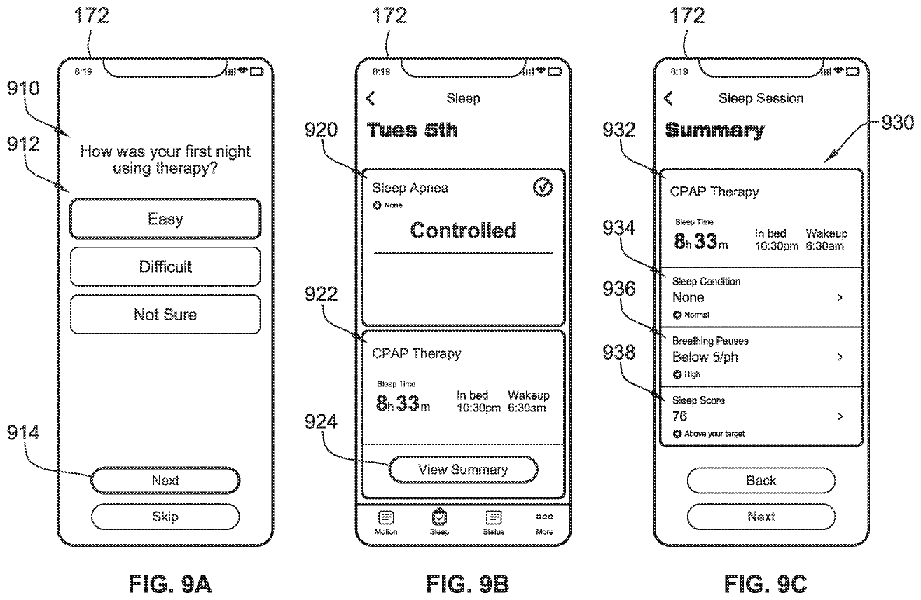

The prompts for the second sleep session (step 607) can be the same as, or similar to, the prompts described above for the first sleep session (step 603), including the first prompt 710 (FIG. 7A), the second prompt 720 (FIG. 7B), the third prompt 730 (FIG. 7C), the fourth prompt 740 (FIG. 7D), or any combination thereof. Referring to FIG. 9A, in some implementations, a fifth prompt 910 containing alphanumeric text prompts the user to indicate feedback associated with use of the respiratory therapy system during the second sleep session. As shown, the fifth prompt 910 includes a plurality of user-selectable elements 912 for prompting the user indicate whether use of the respiratory therapy system was easy, difficult, or if the user is not sure. The user can finalize the selection one of the user-selectable elements 912 by selecting navigation element 914. The second subjective feedback can also include activity information, and can be received in the same or similar manner as described above for step 603 (FIG. 14).

Step 608 of the method 600 includes causing one or more indications associated with at least a portion of the second set of sleep-related parameters (step 606) to be communicated to the user subsequent to the second sleep session. The indication(s) of the determined second set of sleep-related parameters can be communicated to the user via alphanumeric text, images, audio, or any combination thereof using, for example, the user device 170 (FIG. 1). The one or more indications of the second set of sleep-related parameters (step 606) can be the same as the one or more indications of the first set of sleep-related parameters (step 604).

Referring to FIG. 9B, a sleep apnea indication 920 and a therapy indication 922 are displayed on the display device 172 of the user device 170 (FIG. 1). The sleep apnea indication 920 provides information indicative of whether the user experienced sleep apnea (e.g., based on the AHI) during the second sleep session. In the example of FIG. 9B, the user did not experience sleep apnea during the sleep session (e.g., the AHI was less than 5) and the sleep apnea indication 920 indicates that the apnea is controlled. The therapy indication 922 provides information indicative of the use of the respiratory therapy system during the second sleep session including, for example, a total sleep time, an enter bed time, a wake-up time, or any combination thereof. The user can navigate to a summary of the second sleep session be selecting (e.g., clicking or tapping) navigation element 924.

Referring to FIG. 9C, a summary view 930 for the second sleep session is displayed on the display device 172 of the user device 170 (FIG. 1). The summary view 930 includes a plurality of indications 932-938. A first indication 932 is the same as, or similar to, the therapy indication 922 (FIG. 9B) and provides information indicative of the use of the respiratory therapy system during the second sleep session including, for example, a total sleep time, an enter bed time, a wake-up time, or any combination thereof. A second indication 934 is the same as, or similar to, the sleep apnea indication 920 and provides information indicative of whether the user experienced sleep apnea (e.g., based on the AHI) during the second sleep session. A third indication 936 provides information indicative of the AHI for the second sleep session. A fourth indication 938 provides information indicative of a sleep score for the second sleep session and/or whether the determined sleep score meets or exceeds a target sleep score.

In some implementations, steps 605-608 can be repeated for one or more additional sleep sessions subsequent to the second sleep session (e.g., a third sleep session, a fourth sleep session, a tenth sleep session, a one-hundredth sleep session etc.) when the user is using the respiratory therapy system. Determined sleep-related parameters for these additional sleep sessions can be compared to the determined sleep-related parameters for the first sleep session when the user did not use the respiratory therapy system and/or any one of the other additional sleep sessions. Similarly, steps 601-604 can be repeated for one or more additional sleep session subsequent to the first sleep session when the user is not using the respiratory therapy system. For example, steps 605-608 can be repeated for a third sleep session when the user is using the respiratory therapy system, and steps 601-605 can be repeated for a fourth sleep session when the user is not using the respiratory therapy system.

Step 609 of the method 600 includes causing one or more comparisons between at least a portion of the first set of sleep-related parameters and at least a portion of the second set of sleep-related parameters to be communicated to the user. For example, a first one the first sleep-related parameters associated with the first sleep session can be compared to a corresponding one of the second set of sleep-related parameters associated with the second sleep session. The comparison(s) of the determined second set of sleep-related parameters can be communicated to the user via alphanumeric text, images, graphs or charts (e.g., line graphs or plots, bar charts or graphs, etc.), audio, or any combination thereof using, for example, the user device 170 (FIG. 1).

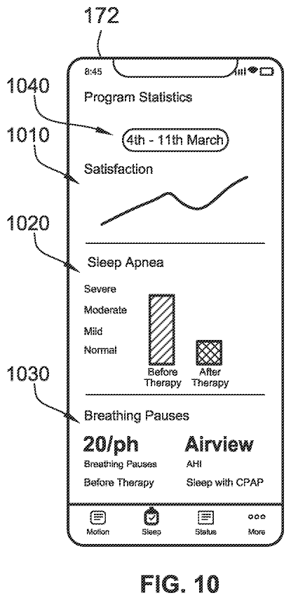

Referring to FIG. 10, a first comparison 1010, a second comparison 1020, and a third comparison 1030 are displayed on the display device 172 of the user device 170 (FIG. 1). The first comparison 1010 is a line plot comparing a sleep satisfaction associated with the first sleep session (e.g., that was provided as part of the first subjective feedback in step 603) with a sleep session satisfaction associated with the second sleep session (e.g., that was provided as part of the second subjective feedback in step 607). As shown, the sleep satisfaction generally improved for the second sleep session when the user was using the respiratory therapy system.

The second comparison 1020 is a bar chart comparing the AHI for the first sleep session and the AHI for the second sleep session. As shown, the comparison 1020 shows that the user experienced severe sleep apnea during the first sleep session (with no respiratory therapy system), but that the user experienced only mild sleep apnea during the second sleep session, demonstrating the effects of using the respiratory therapy system during the second sleep session. The third comparison 1030 includes alphanumeric text providing information about additional ones of the first set of sleep-related parameters associated with the first sleep session and the second set of sleep-related parameters associated with the second sleep session.

A date element 1040 is also displayed along with the first comparison 1010, the second comparison 1020, and the third comparison 1030. By selecting the date element 1040, the user can specify a date range of sleep sessions to include in the comparison (e.g., all sleep sessions between March 4^{th} and March 11^{th}). In this manner, the user can view a comparison of more than two sleep sessions (e.g., three sleep sessions, five sleep sessions, seven sleep sessions, thirty sleep sessions, etc.).

In some implementations, step 609 includes causing an indication associated with an amount of time the user stopped breathing during the first sleep session, the second sleep session, or both, to be communicated to the user (e.g., via the display device 172). For example, the one or more indications can indicate that the user stopped breathing for 10 minutes during the first sleep session (e.g., when not using the respiratory therapy system 120) and did not stop breathing during the second sleep session (e.g., when using the respiratory therapy system 120).

In some implementations, step 609 includes causing one or more indications associated with one or more events experienced during the first sleep session, the second sleep session, or both to be communicated to the user. In such implementations, the one or more indications can be overlaid on at least a portion of a hypnogram (e.g., that is the same as, or similar to, the hypnogram 500 of FIG. 5) associated with the respective sleep session. These indications can aid the user in visualizing the events experienced during sleep. For example, the indications can aid the user in understanding when events (e.g., apneas) are most likely to occur during sleep (e.g., at what times, during what sleep stages, etc.). As described herein, some users of respiratory therapy systems may begin using the system, but remove the system during sleep (e.g., because it is uncomfortable). In some cases, a user may be informed that the user need only use a respiratory therapy system for a certain amount of time (e.g., four hours) to clinically comply with the prescribed use. However, communicating these indications can aid the user in understanding that events are more likely to occur during, for example, after the user has been asleep for four hours, thereby encouraging the user to continue to use the respiratory therapy system during the sleep session. Consequently, these indications can aid the user in improving compliance.

Some users of the respiratory therapy systems described herein (e.g., CPAP systems) find such systems to be uncomfortable, difficult to use, expensive, and/or aesthetically unappealing. Some users of these systems also may not immediately notice any benefits of use after first beginning therapy. As a result, these users may choose not to use their respiratory therapy system as prescribed (e.g., every night), or even completely discontinue use of the respiratory therapy system altogether. Displaying the comparison(s) between the first sleep session (without the respiratory therapy system) and the second sleep session (with the respiratory therapy system) can aid in encouraging the user to continue use the respiratory therapy system for subsequent sleep sessions. For example, the comparison can inform the user that they are receiving a benefit from using the respiratory therapy system, as evidenced by, for example, a lower or improved AHI for the second sleep session, less awakenings during the second sleep session, longer blocks of sleep (e.g., REM sleep) for the second sleep session, improved subjective feedback for the second sleep session, etc. The comparison can also remind the user of the negative symptoms and affects felt following the first sleep session when the user did not use the respiratory therapy system.

Alternatively, in some implementations, the method 600 includes providing a recommendation that the user discontinues use of the respiratory therapy system. For example, the comparison between the determined first set of sleep-related parameters associated with the first sleep session and the second set of sleep-related parameters associated with the second sleep (and/or additional sleep sessions subsequent to the second sleep session) reveals that the user has not benefitted from the respiratory therapy system (e.g., the AHI remains the same or is worse when using the respiratory therapy system), the user may have experienced co-morbid sleep apnea that requires a different treatment or therapy.

In some implementations, the one or more indications that are displayed during step 604 and/or step 609 include information associated with the selected sleep label for the first sleep session, the second sleep session, or both. Further, in some implementations, the one or more comparisons communicated to the user during step 609 can also include information associated with the selected sleep label for the first sleep session, the second sleep session, or both. A comparison between the first sleep label and the second sleep label can aid in selecting product(s) (e.g., respiratory therapy systems or devices) that aid in the user in increasing sleep qualify. For example, if the first sleep label indicates no therapy usage and the second sleep label indicates usage of a respiratory therapy system, a comparison of these sleep labels can aid the user in visualization of the differences in sleep when using therapy (e.g., improvements). As another example, if the first sleep label indicates usage of a first therapy device (e.g., a first user interface) and the second sleep label indicates usage of a second therapy devices (e.g., a second user interface that is different than the first user interface), a comparison of these sleep labels can aid the user is selecting which one of the therapy devices is most effective for improving sleep. Further, the sleep labels can be used to recommend alternative therapy systems or devices (e.g., MRD instead of a CPAP system) or surgery to the user. Further, the sleep labels can be used to recommend complementary devices (e.g., bedding, sleep blankets, pillows, etc.) to the user to improve sleep quality.

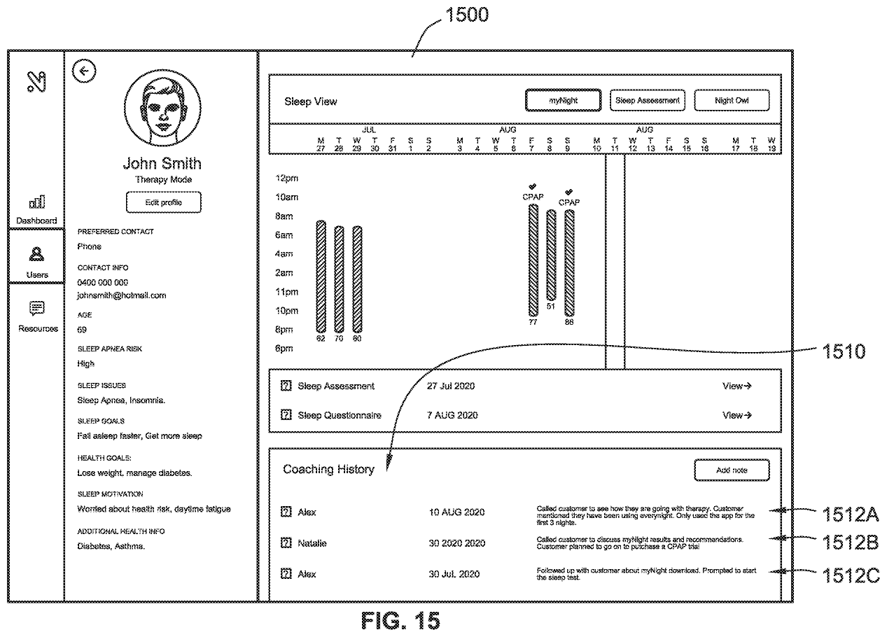

Referring to FIG. 15, in some implementations, the method 600 includes causing a sleep dashboard 1500 to be communicated to the user and/or a third party (e.g., a sleep coach). For example, the sleep dashboard 1500 can be displayed on the display device 172. The sleep dashboard 1500 can be displayed after the first sleep session (e.g., during step 604), after the second sleep session (e.g., during step 608 or step 609), or both. Generally, the sleep dashboard 1500 includes the same or similar types of information as shown in FIGS. 8A-8C, 9A-9C, and 10. In particular, the sleep dashboard 1500 includes coaching information 1510. Generally, the coaching information 1510 includes interactions between the user and a third party (e.g., medical professional such as a physician, therapy system manufacturer or distributor, etc.) regarding the determined sleep-related parameters described herein and/or usage (or lack thereof) of a therapy system (e.g., respiratory therapy system). The coaching information 1510 can include, for example, a first coaching indicator 1512A, a second coaching indicator 1512B, and a third coaching indicator 1512C. These coaching indicators 1512A-1512C provide a summary or overview of the user's progress and can aid in encouraging the user to use a recommended therapy system.

Additionally, in some implementations, the coaching indicators 1512A-1512C can include training programs (e.g., training or instructing the user how to use the respiratory therapy system, generally aiding the user in sleeping, etc.). For example, the training programs can indicate when, and for how long, a device (e.g., the user device 170, the respiratory therapy device 122, etc.) automatically generates one or more lights and/or sounds. In some examples, the training programs cause light of a predetermined color to be emitted at a predetermined time to signal to the user when to wake up and get out of bed. The training programs can be scheduled based on a number of set parameters including a program start time, a program end time or duration, a program frequency or start date(s), or any combination thereof. The training programs can be stored in the memory 114 (FIG. 1).

In some implementations, the systems and methods described herein include an interactive chat assistant software module and interface. Generally, the chat assistant (which can also be referred to as a chat bot) provides automated coaching information (e.g., that is the same as or similar to the coaching information described above) to a user. In particular, the chat assistant can provide information (e.g., coaching information) responsive to an input (e.g., question) from a user. The interactive chat assistant interface can be displayed via the user device 170 described herein to prompt the user to provide an input (e.g., question), for example, using one or more chat windows. The input (e.g., question) from the user can also be received via the user device 170 (e.g., the user can ask questions via alphanumeric text inputted via a keyboard or mouse or verbally using speech to text).

In such implementations, the memory 114 (FIG. 1) includes a chat assistant database storing various images, videos, animations, or other media. Such information in the chat assistant database can aid, for example, in assisting the patient with setting up and configuring a portion of the system 100. For example, the chat assistant database may include images or videos showing a patient how to set up, turn on, and use the respiratory therapy system 120, how to size, adjust, and place the user interface 124, or other help content to reduce the patient's learning curve in using the respiratory therapy system 120. Information in the chat assistant database can be associated or linked to certain inputs (e.g., questions) from the user so that the appropriate information is communicated to the user in response to an input. For example, the user can provide an input with a question (e.g., how do I put on my interface/mask?) and the chat assistant can provide information responding to that question (e.g., images/video with text/audio instructions for putting on the interface/mask). While the chat assistant interface has been described as being automated, in some implementations, the chat assistant interface can allow the user to speak to a live human representative. For example, the chat assistant interface includes an option (e.g., selectable element) to allow the user to choose to speak to a live representative. As another example, the chat assistant can automatically direct the user to a live representative if the chat assistant is unable to answer questions asked by the user.

As described herein, in some implementations, steps 601-604 are performed for a first sleep session in which the user is not using a respiratory therapy system, while steps 605-607 are performed for a second sleep session in which the user is using a respiratory therapy system. In other implementations, steps 601-604 can be repeated one or more times for sleep sessions where the user is not using the respiratory therapy system, then steps 605-609 are performed for one or more sleep sessions in which the user is using a respiratory therapy system. For example, steps 601-604 can be repeated for a first sleep session, a second sleep session, and a third sleep session during which the user does not use a respiratory therapy system. As described herein, the determined sleep-related parameters can be useful in diagnosing the user with a sleep-related disorder such that the user can be prescribed a respiratory therapy system.

Repeating steps 601-604 for multiple sleep sessions without a respiratory therapy system can be advantageous in that the determined sleep-related parameters for the multiple sleep sessions can be evaluated for diagnosing the user with a sleep-related disorder. For example, rather than diagnosing a user based on an AHI for a first sleep session, the AHI for three sleep sessions can be averaged for the diagnosis. More generally, any suitable statistical analysis can be applied to the sleep-related parameters for multiple sleep sessions without usage of a respiratory therapy system to aid in an accurate diagnosis or identification or sleep-related disorders (e.g., removing outliers, averaging, weighed averages, etc.).

While the method 600 has been described herein as including each of steps 601-609, more generally, the method 600 can include any suitable combination of steps 601-609. For example, a first alternative method can include step 601, step 602, step 603, step 605, step 606, step 607, and step 609. As another example, a second alternative method can include step 601, step 602, step 605, step 606, and step 609. Further, while the method 600 has been shown and described herein as occurring in a certain order, more generally, the steps of the method 600 can be performed in any suitable order.

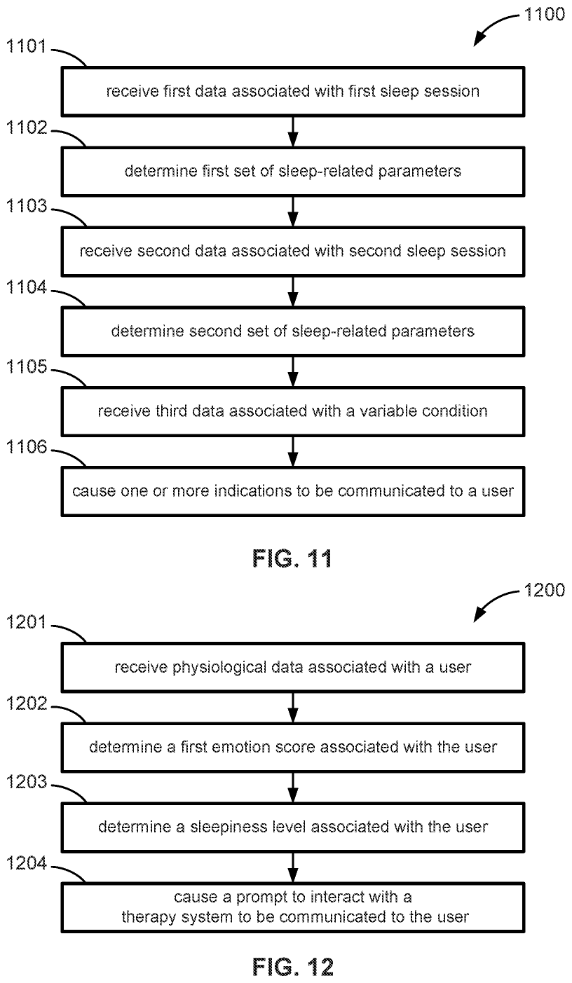

Referring to FIG. 11, a method 1100 according to some implementations of the present disclosure is illustrated. One or more steps of the method 1100 can be implemented using any element or aspect of the system 100 (FIG. 1) described herein.

Step 1101 of the method 1100 includes generating and/or receiving first data associated with a first sleep session of a user. The first data can include, for example, first respiration data associated with the user, first audio data associated with the user, or both. The first respiration data is indicative of is indicative of a first respiration signal of the user during at least a portion of the first sleep session (e.g., at least 10% of the first sleep session, at least 50% of the first sleep session, 75% of the first sleep session, at least 90% of the first sleep session, etc.). The respiration signal is indicative of a respiration rate, a respiration rate variability, a tidal volume, an inspiration amplitude, an expiration amplitude, an inspiration-expiration ratio, etc., or any combination thereof of the user during at least a portion of the first sleep session. The first audio data is reproducible as one or more sounds recorded during the first sleep session (e.g., snoring, choking, labored breathing, etc.). More generally, the first data can include any physiological data associated with the first sleep session of the user.

The first data can be generated by, for example, one or more of the sensors 130 (FIG. 1) described herein. In some implementations, both the first respiratory data and the first audio data are generated by the acoustic sensor 141 (FIG. 1), where the acoustic sensor is coupled to or embedded in user device 170. In other implementations, the first respiratory data is generated by the acoustic sensor 141, whereas the first audio data is generated by a microphone (e.g., that is the same as, or similar to, the microphone 140) that is separate and distinct from the acoustic sensor. The first data can be received by, for example, the electronic interface 119 and/or the user device 170 (FIG. 1) described herein.

Step 1102 of the method 1100 includes determining a first set of sleep-related parameters associated with the first sleep session of the user based at least in part on the first data. For example, the control system 110 of the system 100 (FIG. 1) can analyze the first data (e.g., that is stored in the memory device 114) to determine the first set of sleep-related parameters for the first sleep session. Information describing the determined first set of sleep-related parameters can be stored in the memory device 114 (FIG. 1), for example.

The first set of sleep-related parameters can include, for example, an apnea-hypopnea index (AHI), an identification of one or more events experienced by the user, a number of events per hour, a pattern of events, a total sleep time, a total time in bed, a wake-up time, a rising time, a hypnogram, a total light sleep time, a total deep sleep time, a total REM sleep time, a number of awakenings, a sleep-onset latency, or any combination thereof. In some implementations, the first set of sleep-related parameters can include a sleep score, such as the ones described in International Publication No. WO 2015/006364 and U.S. Patent Publication No. 2016/0151603, which are hereby incorporated by reference herein in its entirety. The first set of sleep-related parameters can include any number of sleep-related parameters (e.g., 1 sleep-related parameter, 2 sleep-related parameters, 5 sleep-related parameters, 50 sleep-related parameters, etc.).

In some implementations, the method 1100 further includes receiving first subjective feedback associated with the first sleep session subsequent to the first sleep session in the same or similar manner as step 603 of the method 600 (FIG. 6) described herein.

Step 1103 of the method 1100 includes receiving second data associated with a second sleep session of the user. The second data can be received by, for example, the electronic interface 119 and/or the user device 170 (FIG. 1) described herein. The second data can be generated using the same sensor as the first data (step 1101), or a different sensor or sensors. In some implementations, the first data (step 1101) and the second data (step 1105) are both generated by the acoustic sensor 141 (FIG. 1). In other implementations, the first data is generated by the acoustic sensor 141 that is coupled to or integrated in the user device 170, while the second data is generated by one or more of the sensors 130 described herein that is coupled to or integrated in the respiratory therapy device 122 (e.g., the pressure sensor 132, the flow rate sensor 134, or both). In still other implementations, the second data is generated by both the acoustic sensor 141 that is coupled to or integrated in the user device 170 and by one or more of the sensors 130 described herein that is coupled or integrated in the respiratory therapy device 122.

The second data is the same as, or similar to, the first data (step 1101) and can include, for example, second respiration data associated with the user, second audio data associated with the user, or both. The second respiration data is indicative of a second respiration signal of the user during at least a portion of the second sleep session (e.g., at least 10% of the second sleep session, at least 50% of the second sleep session, 75% of the second sleep session, at least 90% of the second sleep session, etc.). The respiration signal is indicative of a respiration rate, an inspiration amplitude, an expiration amplitude, an inspiration-expiration ratio, etc., or any combination thereof of the user during at least a portion of the second sleep session. The second audio data is reproducible as one or more sounds recorded during the second sleep session (e.g., snoring, choking, labored breathing, etc.).

The second sleep session is subsequent to the first sleep session. In some implementations, the second sleep session (step 1103) is the next immediate sleep session following the first sleep session (step 1101) (e.g., the first sleep session is a Monday night and the second sleep session is a Tuesday night). In other implementations, there are one or more other sleep sessions between the first sleep session and the second sleep session (e.g., the first sleep session is on a Monday night and the second sleep session is the following Thursday night). The second sleep session can be manually initiated and/or terminated by the user in the same or similar manner as the first sleep session (FIGS. 5A-5C).

Step 1104 of the method 1100 includes determining a second set of sleep-related parameters associated with the second sleep session of the user based at least in part on the second data. For example, the control system 110 can analyze the second data (e.g., that is stored in the memory device 114) to determine the second set of sleep-related parameters for the second sleep session. Information describing the determined second set of sleep-related parameters can be stored in the memory device 114 (FIG. 1), for example. The second set of sleep-related parameters can include, for example, an apnea-hypopnea index (AHI), an identification of one or more events experienced by the user, a number of events per hour, a pattern of events, a sleep score, a total sleep time, a total time in bed, a wake-up time, a rising time, a hypnogram, a total light sleep time, a total deep sleep time, a total REM sleep time, a number of awakenings, a sleep-onset latency, or any combination thereof. The second set of sleep-related parameters (step 1106) can include the same parameters as the first set of sleep-related parameters (step 1102), or different parameters. More generally, the second set of sleep-related parameters can include any number of sleep-related parameters (e.g., 1 sleep-related parameter, 2 sleep-related parameters, 5 sleep-related parameters, 50 sleep-related parameters, etc.).

In some implementations, the method 1100 further includes receiving second subjective feedback associated with the second sleep session subsequent to the second sleep session in the same or similar manner as step 607 of the method 600 (FIG. 6) described herein.

Step 1105 of the method 1100 includes receiving third data associated with a variable condition. The third data can be received by, for example, the electronic interface 119 and/or the user device 170 (FIG. 1) described herein. In some implementations, the third data can be generated using the same sensor as the first data (step 1101) and/or the second data (step 1103) or a different sensor or sensors. In other implementations, the third data can be received via or in response to one or more user inputs. In some implementations, at least a portion of the third data is received (i) prior to the first sleep session, (ii) during at least a portion of the first sleep session, (iii) subsequent to the first sleep session and prior to the second sleep session, (iv) during at least a portion of the second sleep session, (v) subsequent to the second sleep session, or (vi) any combination thereof.

Generally, the variable condition is a condition that varies between the first sleep session and the second sleep session (e.g., varied by the user). Information about the variable condition can provide insights to the user as to how the variable condition affected the user's sleep. The variable condition can be associated with the first sleep session, the second sleep session, or both. In some implementations, the variable condition is associated with usage of a therapy system by the user during the first sleep session, the second sleep session, or both. For example, the user may not use the therapy system (e.g., the respiratory therapy system 120 described herein) during the first sleep session, but may use the therapy system during at least a portion of the second sleep session. In this example, the third data reflects the fact that the user used the therapy system during at least a portion of the second sleep session, but not the first sleep session (e.g., as indicated by one or more sensors that are coupled to or integrated in the respiratory therapy system). As another example, the user may use the therapy system for a first amount of time during the first sleep session and use the therapy system for a second amount of time during the second sleep session. As yet another example, the user may use a first therapy system (e.g., an alternative therapy system, such as MRD) during at least a portion of the first sleep session and a second therapy system during at least a portion of the second sleep session (e.g., respiratory therapy system). In this example, the user can manually provide an indication of usage of the first therapy system and/or second therapy system, or usage of the first therapy system and/or second therapy system could be detected automatically.

In some implementations, the variable condition is associated with a sleep environment condition. The sleep environment condition can be associated with a complementary device or a complementary therapy that makes use of a complementary device. For example, the commentary device can be bedding used by the user during the first sleep session and/or the second sleep session, such as, for example, a pillow, a pillow case, a mattress, a mattress cover, a mattress topper, a sheet, a blanket, or any combination thereof. For example, the user may use a first pillow during at least a portion of the first sleep session, and a second pillow (or no pillow) during at least a portion of the second sleep session. In this example, the third data can be received via or in response to one or more user inputs (e.g., an indication associated with the bedded) or automatically (e.g., by analyzing data from the camera 150 using, for example, an object recognition algorithm). For example, the presence of a complementary device can be detected automatically by communicating with and/or identifying any “smart” devices including, for example, one or more complementary devices such as a bed, a blanket, or a mattress sensor having communication capability (e.g., Wi-Fi or Bluetooth). In this example, the one or more sensors 130 (e.g., camera 150) can be used to scan a complementary device (e.g., a bed) to estimate the type bedding used, for example. Such scanning could include scanning of a QR code and/or an RFID tag, for example. The system could also connect to other devices, such as a thermostat (e.g., a Google Nest™ thermostat), an air purifier, a humidifier, electrically actuated curtains or blinds, a sound machine program, etc., or any combination thereof.

In other implementations, the sleep environment condition is associated with an ambient temperature, an ambient humidity, an ambient lighting condition, a location, or any combination thereof. For example, the first sleep session may be at a first location (e.g., at home), while the second sleep session may be at a second location (e.g., a hotel). In this example, the third data includes information associated with the location, e.g., as provided by one or more inputs from the user or from a sensor (e.g., a GPS or other location-based sensor). As another example, there may be a first ambient temperature during the first sleep session and a second ambient temperature during the second sleep session (e.g., as determined by the temperature sensor described herein). As yet another example, an ambient lighting condition can be modified (e.g., the ambient lighting can be turned on or off, the intensity or brightness of the ambient lighting can be increased or decreased, the color of the ambient lighting can be modified, etc.). As a further example, an ambient sound (e.g., audio, such as music, from one or more speakers or a TV) can be modified (e.g., turned on or off, a volume can be increased or decreased, etc.).

In some implementations, the variable condition is associated with an activity level of the user. For example, the variable condition can be associated with a first activity level of the user prior to the first sleep session, a second activity level of the user subsequent to the first sleep session and prior to the second sleep session, or both. In such implementations, the third data can be generated or obtained from the activity tracker 180 (FIG. 1) described herein. In other implementations, the variable condition is associated with a diet of the user. For example, the variable condition can be associated with caffeine or alcohol intake by the user (e.g., before the first sleep session, before the second sleep session, or both).

Step 1106 of the method 1100 includes causing one or more indications to be communicated to the user. The one or more indications can be communicated to the user via alphanumeric text, images, graphs or charts (e.g., line graphs or plots, bar charts or graphs, etc.), audio, or any combination thereof using, for example, the user device 170 (FIG. 1). The one or more indications can be associated the variable condition, the first sleep session, the second sleep session, or any combination thereof. In some implementations, the one or more indications can also be associated with one the first set of sleep-related parameters, the second set of sleep-related parameters, or both (e.g., in the same or similar manner as step 609 of the method 600). In such implementations, the one or more indications can include a comparison between a first one of the first set of sleep-related parameters for the first sleep session and a second one of the second set of sleep-related parameters for the second sleep session.

Generally, the one or more indications are communicated to the user to illustrate or demonstrate the effect of the variable condition on sleep. For example, communicating an indication associated with the variable condition and communicating one or more indications of a comparison between one or more of the first set of sleep-related parameters and one or more of the second set of sleep-related parameters can aid in demonstrating to the user the effect of the variable condition on sleep, thereby aiding in encouraging the user to vary the variable condition for future sleep sessions to aid in achieving better sleep. For example, if the variable condition is bedding such as a pillow used during the second sleep session but not the first sleep session, a comparison between a sleep-related parameter such as AHI for the first sleep session and the second sleep session can show the user that the pillow improved sleep quality (e.g., by aiding in preventing OSA). In this manner, the indications can provide insights on the effect of one or more variable conditions on the user's sleep.

In some implementations, steps 1103-1106 can be repeated for one or more additional sleep sessions subsequent to the second sleep session (e.g., a third sleep session, a fourth sleep session, a tenth sleep session, a one-hundredth sleep session etc.). Determined sleep-related parameters for these additional sleep sessions can be compared to the determined sleep-related parameters for the first sleep session to further illustrate the effect of the variable condition on sleep over time.

Referring to FIG. 12, a method 1200 according to some implementations of the present disclosure is illustrated. One or more steps of the method 1100 can be implemented using any element or aspect of the system 100 (FIG. 1) described herein.

Step 1201 of the method 1200 includes receiving data associated with a user. The data can include physiological data associated with the user during a sleep session, for example. The data associated with the user can be the same as, or similar to, the first data received during step 601 of the method 600 (FIG. 6) or the first data received during step 1101 of the method 1100 (FIG. 11). The data can be generated by one or more of the sensors 130 (FIG. 1) described herein and received by, for example, the electronic interface 119 and/or the user device 170 described herein.

Step 1202 of the method 1200 includes determining a first emotion score associated with the user based at least in part on the data associated with the user. Generally, the emotion score is indicative of anxiety or stress currently being experienced by the user. For example, the user may experience anxiety, stress, apprehension, discomfort, etc. prior to using the respiratory therapy system 120. Higher levels of stress, anxiety, discomfort, etc. can make it more difficult for the user to fall asleep and/or may prompt the user to abandon using the respiratory therapy system 120. A quantification of the stress or anxiety of the user via the emotion score can be used to suggest or recommend when the user should begin using the respiratory therapy system 120.

An emotion score can be, for example, a numerical value that is on a predetermined scale (e.g., between 1-10, between 1-100, etc.), a letter grade (e.g., A, B, C, D, or F), or a descriptor (e.g., high, low, medium, poor, normal, abnormal, fair, good, excellent, average, below average, above average, needs improvement, satisfactory, etc.). In some implementations, the emotion score is determined relative to a previous emotion score (e.g., the emotion score is better than a prior emotion score (e.g., an emotion score for the prior day), the emotion score is worse than a prior emotion score, the emotions core is the same as a prior emotion score, etc.) or a baseline emotion score (e.g., the emotion score is 50% greater than the baseline emotion score, the emotion score is equal to the baseline emotion score, etc.).

In some implementations, step 1202 includes determining one or more physiological parameters associated with the user, such as, for example, a respiration rate, heart rate, heart rate variability, cardiac waveform, respiration rate, respiration rate variability, respiration depth, a tidal volume, an inspiration amplitude, an expiration amplitude, an inspiration-expiration ratio, perspiration, temperature (e.g., ambient temperature, body temperature, core body temperature, surface temperature, etc.), blood oxygenation, photoplethysmography (e.g., which can be used to measure SpO₂, peripheral perfusion, pulse-rate, other cardiac-related parameters, etc.), pulse transmit time, blood pressure, or any combination thereof.

These physiological parameters can be indicative of emotion or anxiety of the user. For example, hyperventilation, increased respiration rate (e.g., relative to a baseline associated with the user, a baseline for a group of users, normative values, etc.), decrease heart rate variability (e.g., relative to a baseline associated with the user, a baseline for a group of users, normative values, etc.), cardiac arrhythmias, heart rate, and blood pressure (e.g., adjusting for whether the user is hypertensive or non-hypertensive, nocturnal dips in blood pressure, etc.) can be indicative of an increased anxiety level. Conversely, increased heart rate variability (e.g., relative to a baseline associated with the user, a baseline for a group of users, normative values, etc.) can be indicative of a more relaxed state. The emotion score can be determined, at least in part, by scaling or standardizing one or more of the physiological parameters based on previously recorded physiological parameters for the user, previously recorded physiological parameters for a plurality of other users, or both, that are stored in the user profile described above. Alternatively, the emotion score can be determined by scaling the associated physiological parameter(s) with a desired or target value for the parameter(s).

In some implementations, the data received in step 1201 further includes subjective feedback from the user and step 1202 includes determining the emotion score based at least in part on the received subjective feedback. The subjective feedback can include, for example, self-reported user feedback indicative of a current stress or anxiety level of the user in the form of, for example, a descriptive indicator (e.g., high, low, medium, not sure), a numerical value (e.g., on a scale of 1 to 10, where 10 is very stressed and 0 is not stressed at all), etc. The subjective feedback can be received, for example, via the user device 170. In such implementations, the method 1200 can include communicating one or more prompts to the user to solicit the subjective feedback (e.g., via the display device 172 of the user device 170).

In some implementations, step 1202 includes determining the emotion score based at least in part on demographic information associated with the user. The demographic information can include, for information, indicative of an age of the user, a gender of the user, a weight of the user, a body mass index (BMI) of the user, a height of the user, a race of the user, a relationship or marital status of the user, a family history of insomnia, an employment status of the user, an educational status of the user, a socioeconomic status of the user, or any combination thereof. The demographic information can also include medical information associated with the user, such as, for example, including indicative of one or more medical conditions associated with the user, medication usage by the user, or both. The demographic information can be received by and stored in the memory 114 (FIG. 1). The demographic information can be provided manually by the user, for example, via the user device 170 (e.g., via a questionnaire or survey presented through the display device 172). Alternatively, the demographic information can be collected automatically from one or more data sources associated with the user (e.g., medical records).

Step 1203 of the method 1200 includes determining a first sleepiness level associated with the user based at least in part on the data associated with the user. A sleepiness level is generally indicative of the user's fatigue, drowsiness, alertness, and/or awareness, and more generally is indicative of how close the user is to falling asleep. The sleepiness level can be determined and/or expressed in a variety of ways. For example, a sleepiness level can be a scaled value within a predetermined range (e.g., between 1 and 10) where the highest value is indicative of being very sleepy and the lowest is indicative of not being sleepy (or vice versa). Alternatively, the sleepiness level can be expressed using a subjective descriptor (e.g., extremely sleepy, very sleepy, sleepy, neutral, awake, very awake, extremely awake, etc.). Other examples for expressing a sleepiness level include using the Epworth sleepiness scale, the Stanford sleepiness scale, the Karolinska sleepiness scale, etc.

The sleepiness level can be determined based on various types of data or combinations of data. In one example, the sleepiness level can be determined based on physiological data from the EEG sensor 158 (FIG. 1) described herein. In another example, the sleepiness level can be determined using data from the camera 150 (FIG. 1) to determine one or more properties of the eye(s) of the user that are indicative of sleepiness (e.g., measuring a vertical eye opening or eye height, opening of the eyes, closing of the eyes, blinking of the eyes, eye movement, pupil dilation, etc.). In yet another example, the sleepiness data can be determined based on a heart rate of the user, a heart rate variability of the user, a respiration rate of the user, a respiration rate variability of the user, a body temperature of the user, or any combination thereof.

In some implementations, step 1201 includes receiving subjective feedback from the user and step 1202 includes determining the initial sleepiness level based at least in part on the subjective feedback. The subjective feedback can include, for example, a self-reported subjective sleepiness level (e.g., tired, sleepy, average, neutral, awake, rested, etc.). Information associated with or indicative of the feedback from the user can be received, for example, through the external device 170 (e.g., via alphanumeric text, speech-to-text, etc.). In some implementations, the method 1200 includes prompting the user to provide the feedback. For example, the control system 110 can cause one or more prompts to be displayed on the display device 172 of the external device 170 (FIG. 1) that provides an interface for the user to provide the feedback (e.g., the user clicks or taps to enter feedback, the user enters feedback using an alphanumeric keyboard, etc.). The received user feedback can be stored, for example, in the memory device 114 (FIG. 1) described herein.

Step 1204 of the method 1200 includes causing a prompt to interact with a therapy system to be communicated to the user based at least in part on the emotion score, the sleepiness level, or both. As described herein, many users of a respiratory therapy system may not be motivated to use the system as prescribed for a variety of reasons. Generally, the user takes several steps to set up the respiratory therapy system before use, which can take several minutes. This setup process may be another reason that a given user may decide not to use the respiratory therapy system. For example, if the user is too tired or in a bad mood right before going to bed, they may decide to go to sleep rather than setting up and using the respiratory therapy system. Conversely, if the user is alert and in a good mood or motivated, they are more likely to take the time to setup and use the therapy system. For these and other reasons, it would be advantageous to prompt the user to interact (e.g., set up) a therapy system at predetermined time based at least in part on the emotion score and/or the sleepiness level.

For example, in some implementations, the prompt is communicated to the user in response to the first emotion score satisfying a predetermined condition. The predetermined condition can be indicative of the anxiety or stress of the user being at an acceptable level (e.g., such that the user can fall asleep and begin using the respiratory therapy system 120). In other words, when the emotion score satisfies the predetermined condition, the user is sufficiently relaxed such that the user will be more likely to setup and use respiratory therapy system 120. For example, if the emotion score is a numerical value where a higher numerical value indicates higher anxiety or stress, the predetermined condition can be a numerical value that is indicative of anxiety or stress of the user being at an acceptable level. In this example, the emotion score satisfies the predetermined condition if the emotion score is equal to or less than the predetermined condition.

In some implementations, the predetermined condition is determined based at least in part on previously recorded physiological data associated with the user. In such implementations, the predetermined condition can be determined using a machine learning algorithm. The machine learning algorithm can be trained (e.g., using supervised or unsupervised training techniques) with previously recorded physiological data associated with the user such that the machine learning algorithm is configured to determine the predetermined condition. In such implementations, previously recorded physiological data can include corresponding data relating the user's ability to fall asleep, such as, for example, sleep onset latency, wake-after-sleep onset, sleep efficiency, fragmentation index, time to go to bed, total time in bed, total sleep time, or any combination thereof. The previously recorded data for training the machine learning algorithm can also include subjective feedback from the user, as described herein.

For example, in some implementations, the prompt is communicated to the user in response to the first sleepiness level satisfying a predetermined condition or threshold. The predetermined condition or threshold can be indicative of the sleepiness or alertness of the user being at an acceptable level (e.g., such that the can effectively set up the respiratory therapy system 120). In other words, when the first sleepiness level satisfies the predetermined condition, the user is sufficiently alert such that the user will be more likely to setup and use respiratory therapy system 120. For example, if the sleepiness level is a numerical value where a higher numerical value indicates more fatigue and less alertness, the predetermined condition can be a numerical value that is indicative of anxiety or stress of the user being at an acceptable level. In this example, the emotion score satisfies the predetermined condition if the sleepiness level is equal to or less than the predetermined condition.

In some implementations, a predicted fall asleep time can be determined based at least in part on the determined sleepiness level. For example, the predicated fall asleep time may indicate that that the user is likely to fall asleep within a predetermined amount of time (e.g., within 1 minute, within 5 minutes, within 15 minutes, within 1 hour, within 3 hours, etc.) or a range of times (e.g., in between about 5 minutes and 15 minutes, in between about 15 minutes and 30 minutes, etc.). The predicted fall asleep time can be determined, for example, using a trained machine learning algorithm (e.g., that is trained using prior data from one or more users to receive as an input the sleepiness level and determine as an output the predicted fall asleep time). In some implementations, the user may be consuming (e.g., watching, listening to, etc.) media prior to initiating a sleep session. In such implementations, the methods and systems herein can be configured to cause the media to cease playback or modify one or more parameters of the media (e.g., volume, brightness, etc.) based on the predicted fall asleep time.

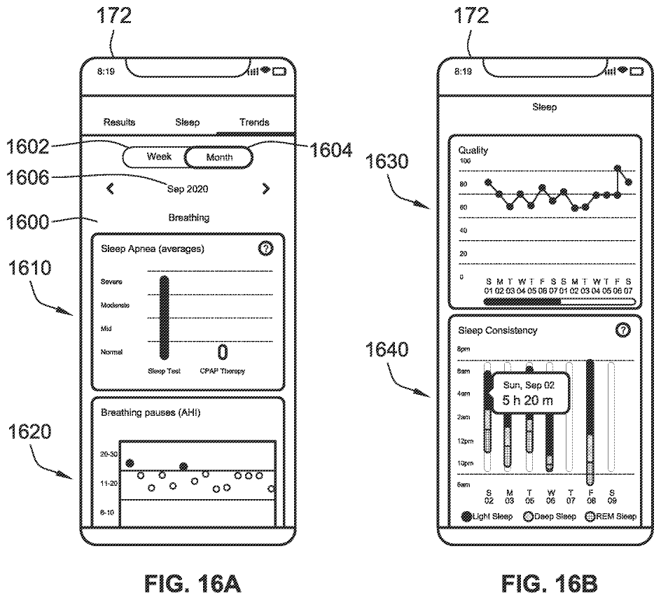

Referring to FIGS. 16A and 16B, one or more trend views can be displayed according to some implementations of the present disclosure (e.g., as part of any of the methods disclosed herein). For example, such trend views can be displayed using the display device 172. Referring to FIG. 16A, a trend view 1600 is displayed on the display device 172. The trend view 1600 includes a week filter 1602 and a month filter 1604. The user can select (e.g., click or tap) the week filter 1602 view trends over the course of a week. The user can select (e.g., click or tap) the month filter 1604 to view trends over the course of a month. In the example shown in FIG. 16A, the month filter 1604 is selected. The trend view also includes a month/week selector 1606, which allows the user to filter by a specific week or month (e.g., by clicking or tapping arrows or other selectable elements). For example, if the month filter 1604 is selected, the user can use the month/week selector 1606 to choose a month (e.g., September 2020).

The trend view 1600 includes a sleep apnea plot 1610 and a breathing plot 1620. The sleep apnea chart 1610 is generally indicative of an average sleep apnea severity (e.g., severe, moderate, mild, normal (no sleep apnea)) when the user did not use the respiratory therapy system (e.g., labeled sleep test) and when the user did use the respiratory therapy system (e.g., labeled CPAP therapy). The sleep apnea plot 1610 can further aid in visualizing the advantages of using the respiratory therapy system and can thus further aid in encouraging or promoting usage of the respiratory therapy system. The breathing pause plot 1620 is generally indicative of breathing pauses (e.g., expressed using AHI along the y-axis) over the selected time period (e.g., month or week).

Referring to FIG. 16B, the trend view 1600 includes a sleep quality plot 1630 and a sleep consistency plot 1640. The sleep quality plot 1630 is generally indicative of sleep quality over the selected time period (e.g., month, week, etc.). Sleep quality can be expressed, for example, using one of the sleep scores described herein (e.g., using a line chart or other suitable graph, chart, or plot). Additionally, the user can scroll (e.g., by swiping left or right) to see additional day(s) and/or week(s) within the specified time range. The sleep consistency plot 1640 is generally indicative of sleep consistency over the selected time period (e.g., month, week, etc.). Sleep quality can be shown, for example, on a plot with the y-axis being a time (e.g., between 8 am and 10 pm) with lines or strips for each day with lines or strips representing sleep stages or states (e.g., using different colors for different sleep stages or states, such as a first color for light sleep, a second color for deep sleep, and a third color for REM sleep). In some implementations, each strip or line in the sleep consistency plot is selectable by a user (e.g., by clicking or tapping) to display additional information (e.g., total sleep time in hours and minutes, the date, etc.).

One or more elements or aspects or steps, or any portion(s) thereof, from one or more of any of claims 1-114 below can be combined with one or more elements or aspects or steps, or any portion(s) thereof, from one or more of any of the other claims 1-114 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
receiving first data associated with a first sleep session of a user, the first data including (i) first respiration data associated with the user, (ii) first audio data reproducible as one or more sounds recorded during the first sleep session, or (iii) both (i) and (ii), wherein the user did not use a respiratory therapy system during the first sleep session;
determining a first set of sleep-related parameters associated with the first sleep session of the user based at least in part on the first data;
receiving second data associated with a second sleep session of the user, the second data including (i) second respiration data associated with the user, (ii) second audio data reproducible as one or more sounds recorded during the second sleep session, or (iii) both (i) and (ii), wherein the user used the respiratory therapy system during at least a portion of the second sleep session;
determining a second set of sleep-related parameters associated with the second sleep session of the user based at least in part on the second data; and
causing one or more indications associated with the first sleep session, the second sleep session, or both, to be communicated to the user, via a user device, subsequent to the second sleep session to aid in encouraging the user to use the respiratory therapy system.

2. The method of claim 1, wherein the one or more indications further include a recommendation to discontinue use of the respiratory therapy system.

3. The method of claim 1, wherein the one or more indications include (i) an indication associated with at least a portion of the first set of sleep-related parameters, (ii) an indication associated with at least a portion of the second set of sleep-related parameters, or (iii) both (i) and (ii).

4. The method of claim 1, wherein the first set of sleep-related parameters and the second set of sleep-related parameters includes an apnea-hypopnea index (AHI), an identification of one or more events experienced by the user, a number of events per hour, a pattern of events, a sleep score, a total sleep time, a total time in bed, a wake-up time, a rising time, a hypnogram, a total light sleep time, a total deep sleep time, a total REM sleep time, a number of awakenings, a sleep-onset latency, or any combination thereof.

5. The method of claim 4, wherein the one or more indications include an indication of a first AHI for the first sleep session and an indication of a second AHI for the second sleep session.

6. The method of claim 4, wherein the one or more events include snoring, apneas, central apneas, obstructive apneas, mixed apneas, hypopneas, a mask leak, a restless leg, a sleeping disorder, choking, labored breathing, an asthma attack, an epileptic episode, a seizure, or any combination thereof.

7. The method of claim 1, further comprising:
receiving, from the user, first subjective feedback associated with the first sleep session; and
receiving, from the user, second subjective feedback associated with the second sleep session.

8. The method of claim 7, further comprising:
prompting the user to provide the first subjective feedback subsequent to the first sleep session and prior to the second sleep session; and
prompting the user to provide the second subjective feedback subsequent to the second sleep session.

9. The method of claim 7, wherein the first subjective feedback includes a subjective sleepiness level subsequent to the first sleep session, a subjective sleepiness level prior to the first sleep session, a subjective sleep satisfaction rating for the first sleep session, or any combination thereof.

10. The method of claim 7, wherein the one or more indications include (i) an indication of at least a portion of the first subjective feedback for the first sleep session, (ii) an indication of at least a portion of the second subjective feedback for the second sleep session, or (iii) both (i) and (ii).

11. The method of claim 1, wherein the first set of sleep-related parameters includes an identification of one or more events experienced by the user during the first sleep session, and wherein the method further comprises causing a portion of the first audio data that is associated with the one or more events experienced by the user during the first sleep session to be communicated to the user via a speaker subsequent to the first sleep session.

12-13. (canceled)

14. The method of claim 1, wherein the first data, the second data, or both is generated by one or more sensors, the one or more sensors including an acoustic sensor, a microphone, a speaker, or any combination thereof.

15. (canceled)

16. The method of claim 1, wherein the one or more sensors includes (i) an acoustic sensor having a first microphone and a speaker and (ii) a second microphone, and wherein the first respiration data and the second respiration data are generated by the acoustic sensor and the first audio data and the second audio data are generated by the second microphone.

17-20. (canceled)

21. The method of claim 1, wherein the one or more indications include a comparison between a first one of the first set of sleep-related parameters for the first sleep session and a second one of the second set of sleep-related parameters for the second sleep session.

22. The method of claim 21, wherein the first one of the first set of sleep-related parameters is a first AHI and the second one of the second set of sleep-related parameters is a second AHI.

23. (canceled)

24. The method of claim 7, wherein the one or more indications include a comparison between a portion of the first subjective feedback for the first sleep session and a portion of the second subjective feedback for the second sleep session, wherein the portion of the first subjective feedback includes a first subjective sleep satisfaction for the first sleep session and the portion of the second subjective feedback includes a second subjective sleep satisfaction for the second sleep session.

25-26. (canceled)

27. The method of claim 1, further comprising determining whether the user experienced co-morbid insomnia sleep apnea (i) during the first sleep session based at least in part on the first data, (ii) during the second sleep session based at least in part on the second data, or (iii) both (i) and (ii).

28. The method of claim 1, wherein the first respiration data is indicative of a first respiration signal of the user during at least a portion of the first sleep session and the second respiration data is indicative of a second respiration signal of the user during at least a portion of the second sleep session.

29. The method of claim 28, further comprising causing (i) a representation of at least a portion of the first respiration signal to be displayed on the display device subsequent to the first sleep session, (ii) a representation of at least a portion of the second respiration signal to be displayed on the display device subsequent to the second sleep session, or (iii) both (i) and (ii).

30. The method of claim 29, further comprising causing (i) an indication of one or more events experienced by the user during the first sleep session to be overlaid on the displayed representation of the first respiration signal, (ii) an indication of one or more events experienced by the user during the second sleep session to be overlaid on the displayed representation of the second respiration signal, or (iii) both (i) and (ii).

31-41. (canceled)

42. A system comprising:
a memory storing machine-readable instructions; and
a control system including one or more processors configured to execute the machine-readable instructions to:
receive first data generated by one or more sensors and associated with a first sleep session of a user, wherein the user did use a respiratory therapy system during the first sleep session;
determine a first set of sleep-related parameters associated with the first sleep session for the user based at least in part on the first data;
receive, from the one or more sensors, second data associated with a second sleep session of the user, wherein the user interface of the respiratory therapy system engaged the user during at least a portion of the second sleep session;
determine a second set of sleep-related parameters associated with the second sleep session for the user based at least in part on the second data; and
cause one or more indications associated with the first sleep session, the second sleep session, or both to be communicated to the user via the display of the user device subsequent to the second sleep session.

43. The system of claim 42, further comprising the respiratory therapy system, wherein the respiratory therapy system includes:
a respiratory device configured to supply pressurized air; and
a user interface coupled to the respiratory device via a conduit, the user interface being configured to engage a user and aid in directing the supplied pressurized air to an airway of the user;

44. The system of claim 42, wherein at least one of the one or more sensors is coupled to or integrated in the respiratory therapy system or (ii) the user device.

45. (canceled)

46. The system of claim 42, wherein the first data associated with the first sleep session is generated by a first sensor of the one or more sensors and the second data associated with the second sleep session is generated by a second sensor of the one or more sensors that is separate and distinct from the first sensor.

47. The system of claim 42, wherein the one or more sensors include a microphone, a speaker, an acoustic sensor, a pressure sensor, a flow rate sensor, or any combination thereof.

48-50. (canceled)

51. A method comprising:
receiving, from one or more sensors, first data associated with a first sleep session of a user, the first data including (i) first respiration data associated with the user, (ii) first audio data reproducible as one or more sounds recorded during the first sleep session, or (iii) both (i) and (ii), wherein the user did not use a respiratory therapy system during the first sleep session;
determining a first set of sleep-related parameters associated with the first sleep session of the user based at least in part on the first data, the first set of sleep-related parameters including a first apnea-hypopnea index (AHI) for the first sleep session;
determining a first sleep condition for the first sleep session based at least in part on the first AHI;
receiving, from the one or more sensors, second data associated with a second sleep session of the user, the second data including (i) second respiration data associated with the user, (ii) second audio data reproducible as one or more sounds recorded during the second sleep session, or (iii) both (i) and (ii), wherein the user used the respiratory therapy system during at least a portion of the second sleep session;
determining a second set of sleep-related parameters associated with the second sleep session of the user based at least in part on the second data, the second set of sleep-related parameters including a second AHI for the second sleep session;
determining a first sleep condition for the second sleep session based at least in part on the second AHI;
causing one or more indications of (i) the first sleep condition, (ii) the second sleep condition, or (iii) both (i) and (ii) to be communicated to the user via a user device subsequent to the first sleep session.

52-114. (canceled)
